SEQUENCE LISTING (6 sequences)

SEQ ID NO: 1 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 2 - protein, 249 aa, Mus musculus
MAARRSQRRRGRRGEPGTALLAPLVLSLGLALACLGLLLVVVSLGSWATLSAQEPSQEELTAEDRREPPELNPQTEESQDVVPFLEQLVRPRRSAPKGRKARPRRAIAAHYEVHPRPGQDGAQAGVDGTVSGWEETKINSSSPLRYDRQIGEFTVIRAGLYYLYCQVHFDEGKAVYLKLDLLVNGVLALRCLEEFSATAASSPGPQLRLCQVSGLLPLRPGSSLRIRTLPWAHLKAAPFLTYFGLFQVH

SEQ ID NO: 3 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 4 - protein, 129 aa, Mus musculus
MAPGWPRSLPQILVLGFGLVLMRAAAGEQAPGTSPCSSGSSWSADLDKCMDCASCPARPHSDFCLGCAAAPPAHFRLLWPILGGALSLVLVLALVSSFLVWRRCRRREKFTTPIEETGGEGCPGVALIQ

SEQ ID NO: 5 - protein, 447 aa, Artificial sequence
  Synthetic polypeptide
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 218 aa, Artificial Sequence
  Synthetic polypeptide
DIVLTQSPASLAVSLGQRATISCRASKSVSTSSYSYMHWYQQKPGQPPKLLIKYASNLESGVPARFSGSGSGTDFSLNIHPMEEDDTAMYFCQHSRELPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2012/0020913 A1
(43) Pub. Date: Jan. 26, 2012

(54) METHODS FOR PANCREATIC TISSUE REGENERATION

(75) Inventor: Linda Burkly, West Newton, MA (US)

(73) Assignee: BIOGEN IDEC MA INC., Cambridge, MA (US)

(21) Appl. No.: 13/146,551

(22) PCT Filed: Jan. 29, 2010
(86) PCT No.: PCT/US2010/022610

§ 371 Date: Oct. 7, 2011

Related U.S. Application Data
(60) Provisional application No. 61/148,701, filed on Jan. 30, 2009.

(51) Int. Cl. A61K 38/19; A61P 3/10; A61P 1/18; A61K 39/395; A61K 35/12

(52) U.S. Cl. 424/85.1; 424/172.1; 424/93.7

(57) ABSTRACT

Disclosed are methods of expanding populations of pancreatic cells or inducing the generation of pancreatic progenitor cells in a subject or in culture using a therapeutically effective amount of a TWEAK receptor agonist. These methods may be used to treat diseases or conditions where enhancement of pancreatic progenitor cells for cell replacement therapy is desirable, including, e g, diabetes and conditions that result in loss of all or part of the pancreas

This invention involves methods for expanding populations of pancreatic cells and inducing regeneration of pancreatic tissue.

The regenerative process of pancreatic cells is of particular interest because of the inadequate number of insulin-producing beta cells in patients with diabetes and because of the possibility that pancreatic cancer may arise from the uncontrolled growth of pancreatic progenitor cells. The mechanisms that have been proposed to produce new beta cells include the replication of preexisting beta cells and neogenesis of beta cells, wherein insulin-positive cells differentiate from progenitor cells. In the neogenesis mechanism, it has been suggested that differentiated duct epithelial cells act as the pancreatic progenitor cells (Bonner-Weir et al., Pediatric Diabetes 5:15-22 (2005); Sharma et al., Diabetes 48:507-513 (1999)). Recent evidence in mice strongly supports this hypothesis: differentiated ductal cells genetically marked with a duct-specific carbonic anhydrase reporter gene act as pancreatic progenitors that give rise to both new islets and pancreatic acini (digestive enzyme-secreting tissue) both after birth and after injury (Inada et al., Proc. Nat. Acad. Sci. USA 105(50):19915-9 (2008)). The neogenesis mechanism may also involve resident progenitor cells that can express lineage specification markers, including, for example, Ngn3 and Pdx-1, which direct their differentiation of the cells into insulin-positive cells. Evidence supporting the ability of Ngn3-positive progenitors to give rise to insulin-positive cells was recently reported in a model of pancreatic ductal ligation injury (Xu, et al. Cell 132:197 (2008)). Thus, it is possible that the Ngn3- and/or Pdx1-positive progenitors are derived from dedifferentiated duct epithelial cells that are intermediates in the process of neogenesis of insulin-positive cells. Alternatively, progenitors capable of expressing Ngn3 and/or Pdx1 may reside in the adult pancreas.

Diabetes mellitus is a disease affecting approximately 7.5 million people in the United States. The underlying cause of this disease is diminished or absent insulin production by beta cells in the Islets of Langerhans in the pancreas. A major goal in diabetes therapy is to recapture the ability to regenerate or replace insulin-producing beta cells. Although transplantation of Islet of Langerhans cells can be an effective treatment, application of this therapy is limited by the short supply of islets that can be obtained through organ donation. Two alternate means of obtaining replacement beta cells for treatment of either type 1 or type 2 diabetes are the regeneration of beta cells from endogenous precursors in vivo and the expansion and differentiation of beta cell precursors in vitro for transplant therapy. Despite great potential, both approaches are limited by the lack of factors that can effectively expand and differentiate beta cell precursors. Cell transplant-based therapies face particularly significant difficulties in ex vivo expansion of rare progenitor cells in an undifferentiated state and their tendency towards undergoing cell death upon delivery in vivo.

### SUMMARY OF THE INVENTION

The invention relates at least in part to a discovery that the TNF family member TWEAK is capable of expanding populations of human and rodent pancreatic cells and inducing the appearance of endocrine lineage committed progenitor cells in the pancreas. Accordingly, the invention provides methods for regenerating pancreatic tissue and expanding populations of pancreatic cells in vivo and in vitro using agonists of the TWEAK receptor (TWEAK-R). These methods may be used to treat diseases or conditions where enhancement of pancreatic progenitor cells for cell replacement therapy is desirable, including, e.g., diabetes and conditions that result in loss of all or part of the pancreas.

Aspects of the invention encompass the use of a TWEAK-R agonist for inducing regeneration of pancreatic tissue in a subject. In exemplary embodiments, the pancreatic tissue is pancreatic islet tissue. In some embodiments, the pancreatic islet tissue comprises islet beta cells.

In exemplary embodiments, the subject has lost pancreatic tissue. In some embodiments, the subject has diabetes, and the method comprises administering to the subject an amount of a TWEAK-R agonist effective to induce the regeneration of insulin-secreting pancreas tissue. The diabetes can be type 1 diabetes or type 2 diabetes.

In some embodiments, the subject has undergone removal of pancreatic tissue. In exemplary embodiments, the subject has undergone removal of cancerous pancreatic tissue.

In some embodiments, the subject has received a cell or tissue transplant in the pancreas, wherein the transplant comprises progenitor cells capable of differentiating into pancreatic cells. In exemplary embodiments, the transplant comprises pancreatic progenitor cells or cells capable of dedifferentiating into pancreatic progenitor cells. The pancreatic progenitor cells may be CK-, Ki-67-, Pdx1- and/or Ngn3-positive cells. The pancreatic progenitor cells may be pancreatic ductal epithelial cells or ductal adjacent cells. In exemplary embodiments, the transplant consists essentially of pancreatic ductal epithelial cells.

In some embodiments, the pancreatic progenitor cells are embryonic stem cells, adult stem cells, totipotent stem cells or pluripotent stem cells that are capable of differentiating into pancreatic cells. In some embodiments, the methods comprise administering the transplant prior to administering a TWEAK-R agonist. In some embodiments, the transplant and the TWEAK-R agonist are administered simultaneously.

Aspects of the invention encompass methods of treating diabetes in a subject, comprising administering to a subject with diabetes i) a cell or tissue transplant comprising progenitor cells capable of differentiating into pancreatic cells, and ii) a therapeutically effective amount of a TWEAK-R agonist sufficient to induce regeneration of pancreatic tissue.

Aspects of the invention further encompass methods of regenerating pancreatic tissue in a subject who has undergone a partial pancreatectomy, comprising administering to the subject an amount of a TWEAK-R agonist effective for inducing regeneration of pancreatic progenitor cells in the subject, wherein the progenitor cells reside in the subject or are transplanted into the subject.

In certain embodiments, the methods of the invention comprise co-administering to the subject a TWEAK-R agonist and an anti-inflammatory agent or other immunomodulatory agent, for example to inhibit the underlying autoimmune response in patients with Type 1 diabetes. Exemplary immunomodulatory agents include, but are not limited to, anti-CD3 monoclonal antibody and Rituxan. The immunomodulatory agent can be administered at the same time or subsequent to administration of the TWEAK-R agonist.

In some embodiments, the TWEAK-R agonist is administered in or near the pancreas or pancreatic region. In exemplary embodiments, the subject is a mammal. In some embodiments, the subject is a human.

Aspects of the invention encompass methods for expanding a population of pancreatic cells, comprising contacting a population of pancreatic cells comprising at least one pancreatic progenitor cell, or at least one cell capable of dedifferentiating into a pancreatic progenitor cell, with a TWEAK-R agonist to obtain an expanded population of pancreatic cells. In exemplary embodiments, the population of pancreatic cells consists essentially of pancreatic progenitor cells. In some embodiments, the pancreatic progenitor cells are pancreatic ductal epithelial cells and/or ductal adjacent cells. In some embodiments, the pancreatic progenitor cells are CK-, Ki-67-, Pdx1-, and/or Ngn3-positive cells. In some embodiments, the population of pancreatic cells consists essentially only of pancreatic ductal epithelial cells. In some embodiments, the expanded population of pancreatic cells comprises an expanded population of insulin-positive cells. In some embodiments, the expanded population of pancreatic cells comprises an expanded population of Ki-67-, Pdx1-, and/or Ngn3-positive cells.

In exemplary embodiments, the pancreatic cells tare expanded in vitro. In some embodiments, the population of pancreatic cells was obtained from a subject. In some embodiments, a TWEAK-R agonist is administered to the pancreatic cells obtained from a subject in vitro. In exemplary embodiments, the population of pancreatic cells is obtained from a population of essentially essentially pancreatic ductal epithelial cells. In other embodiments, the population of pancreatic cells is obtained from a population of non-pancreatic cells that differentiate into pancreatic cells. In some embodiments, the population of essentially non-pancreatic cells comprises one or more of totipotent stem cells, pluripotent stem cells, embryonic stem cells, and adult stem cells.

Aspects of the invention further encompass methods of treating diabetes comprising the steps of a) culturing progenitor cells or cells capable of dedifferentiating into pancreatic progenitor cells, in vitro, wherein said progenitor cells or cells capable of dedifferentiating into pancreatic progenitor cells are pancreatic progenitor cells isolated from a subject with diabetes, totipotent stem cells, pluripotent stem cells, adult stem cells, and/or embryonic stem cells; b) inducing the proliferation of said cultured progenitor cells with an effective amount of a TWEAK-R agonist to generate an expanded population of said progenitor cells; and c) transplanting said expanded population of progenitor cells into the pancreas of the subject with diabetes; wherein insulin-producing pancreatic cells are regenerated in the subject from said transplanted progenitor cells. In some embodiments, the subject with diabetes is a mammal. In some embodiments, the subject with diabetes is a human.

In exemplary embodiments of the invention, the TWEAK-R agonist is selected from the group consisting of: TWEAK, a TWEAK analog, a TWEAK mimetic, and an agonistic TWEAK-R antibody.

In some embodiments, the TWEAK-R agonist is TWEAK. In some embodiments, TWEAK is a polypeptide with the sequence of SEQ ID NO:1 or SEQ ID NO:2. In exemplary embodiments, TWEAK is a polypeptide with an amino terminus at any position between amino acids 46 and 104 of SEQ ID NO:1 and a carboxy terminus at amino acid 249 of SEQ ID NO:1. In some embodiments, TWEAK comprises amino acids 46 to 249 of SEQ ID NO:1. In further embodiments, TWEAK comprises amino acids 104 to 249 of SEQ ID NO:1. In yet further embodiments, TWEAK is a polypeptide with an amino terminus at any position between amino acids 46 and 104 of SEQ ID NO:2 and a carboxy terminus at amino acid 249 of SEQ ID NO:2.

In exemplary embodiments, the TWEAK-R agonist is a TWEAK analog. In some embodiments, the TWEAK analog is a polypeptide that is at least 80% identical to SEQ ID NO:1. In some embodiments, the TWEAK analog is a polypeptide that is at least 90% identical to SEQ ID NO:1.

In exemplary embodiments, the TWEAK analog is a TWEAK fusion protein. In some embodiments, the TWEAK fusion protein comprises the polypeptide of SEQ ID NO:1 and an Fc portion of an immunoglobulin.

In further exemplary embodiments, the TWEAK-R agonist is an agonistic TWEAK-R antibody. In some embodiments, the agonistic antibody is a monoclonal antibody. In some embodiments, the agonistic antibody is a chimeric antibody.

### BRIEF DESCRIPTION OF DRAWINGS

FIG. 1 depicts pancreatic tissue sections from mice overexpressing TWEAK (right panels) or a control protein (left panels) from an adenoviral vector. Hematoxylin and eosin- (H&E) stained sections are shown in the top panels, and sections stained for the proliferation marker Ki-67 are shown in the bottom panels.

FIG. 2, in the left panel, shows the high frequency of expression of TWEAK-R in pancreatic adenocarcinomas, as measured by immunohistochemical staining of a human pancreatic tumor tissue microarray. The middle and right panels show TWEAK-R staining in pancreatic tumor tissue samples.

FIG. 3 depicts a representative mouse pancreatic H&E-stained section showing the structure of pancreatic tissue, with ducts, islets, blood vessels, and acini (labeled) visible in cross-section.

FIG. 4 shows representative pancreatic tissue sections from mice after treatment with control protein P1.17 or TWEAK. FIGS. 4A and 4B show Ki-67-stained sections from mice three days after treatment with P1.17 (FIG. 4A) or TWEAK (FIG. 4B). FIGS. 4C and 4D show sections co-immunostained for ductal epithelial marker CK and Ki-67 after treatment with control protein (FIG. 4C) or TWEAK (FIG. 4D) for four days.

FIG. 5 shows representative pancreas sections from mice after 4 days of treatment either with control protein P1.17 (left), Fc-TWEAK under the acute treatment regimen (middle), or Fc-TWEAK under the chronic treatment regimen (right), immunostained for the proliferation marker Ki-67. All results shown are representative of four mice per treatment group.

FIG. 6 shows a plot of the percentage of duct epithelial cells that are Ki-67-positive in the pancreas sections of mice administered P1.17 or Fc-TWEAK under the chronic treatment regimen.

FIG. 7 shows the percentage of duct epithelial cells that are Ki-67-positive in the pancreas sections of mice administered P1.17 or Fc-TWEAK under the acute treatment regimen.

FIG. 8 depicts the percentage of cells in ductal adjacent regions that are Ki-67-positive in the pancreas sections of mice under chronic Fc-TWEAK treatment.

FIG. 9 shows the percentage of cells in ductal adjacent regions that are Ki-67-positive in the pancreas sections of mice under acute Fc-TWEAK treatment.

FIG. 10 shows pancreatic serial sections from control protein-treated mice (FIGS. 10A and 10C) and TWEAK-treated mice (FIGS. 10B and 10D), stained for CD3 and F4/80.

FIG. 11 shows the number of Ki-67-positive ductal cells (FIG. 11A) or ductal adjacent cells (FIG. 11B) counted in pancreas sections from TWEAK-R KO and wild type mice treated with control protein P1.17 or TWEAK twice per week for a duration of three or ten days. FIG. 11D shows TWEAK-R immunofluorescent staining in a pancreas section from a mouse treated with TWEAK for three days (isotype control is shown in FIG. 11C).

FIG. 12 depicts representative pictures showing increased numbers of human ductal cells 24 hours and 96 hours following introduction of 20 ng/ml or 50 ng/ml of Fc-TWEAK as compared to cells treated with control protein P1.17.

FIG. 13 depicts the dynamic expression of Ngn3 and Pdx1 during mouse embryonic development.

FIG. 14 shows Ngn3-stained pancreatic tissue from mice after 4 days chronic Fc-TWEAK treatment. Staining for Ngn3 in duct-adjacent cells is highlighted in circles.

FIG. 15 shows pancreatic tissue from mice after 4 days of acute Fc-TWEAK treatment, stained for Ngn3, ductal epithelial marker CK, and DAPI.

FIG. 16 shows the frequency of Ngn3-positive cells in pancreatic tissue taken from mice after four days of either control Ig treatment (injection with P1.17) or treatment with Fc-TWEAK under either the acute or chronic dosing regimen. Frequency of Ngn3-positive cells is calculated by dividing the number of Ngn3-positive cells by the total cell numbers on a single section, as determined by Aperio Software.

FIG. 17 shows staining of Pdx1 in pancreatic ductal epithelial cells after four days of acute Fc-TWEAK treatment (right panel, arrows) or control protein P1.17 treatment (left panel).

FIG. 18 shows serial sections of the pancreas stained for TWEAK-R (Fn14; FIGS. 18A and 18C) or CK, insulin (INS), and DNA (DAPI) (FIGS. 18B and 18D) following partial pancreatectomy. Serial sections of pancreas from sham-operated mice (FIGS. 18E and 18F) and from mice four days after partial pancreatectomy (FIGS. 18G and 18H) are shown, stained with T-cell marker CD3 (FIGS. 18E and 18G) and macrophage marker F4/80 (FIGS. 18F and 18H).

FIG. 19 shows regeneration foci in pancreas sections from mice treated with TWEAK for 18 days.

FIG. 20 shows the percentage of regenerating foci at early (“young”), intermediate, and mature stages in wild type mice (white bars) and TWEAK-R KO mice (black bars) four days after partial pancreatectomy.

FIG. 21 shows pancreatic sections costained for CK and Ki-67 from wild type (A) and TWEAK-R KO (B) mice four days after partial pancreatectomy.

### DETAILED DESCRIPTION

The invention provides methods for regenerating pancreatic tissue and expanding populations of pancreatic cells in vitro or in vivo using a therapeutically effective amount of an agonist of the TWEAK receptor (TWEAK-R). These methods may be used to treat diseases or conditions where enhancement of pancreatic progenitor cells for cell replacement therapy is desirable, including, e.g., diabetes and conditions that result in loss of all or part of the pancreas.

The invention relates at least in part to a discovery that the TNF family member TWEAK is capable of expanding populations of human and rodent pancreatic cells and inducing the appearance of endocrine lineage committed progenitor cells in the pancreas. TWEAK is therefore involved in one or more of i) dedifferentiating pancreatic cells, e.g., pancreatic duct epithelial cells, into cells that are capable of differentiating into cells of a committed pancreatic cell lineage; ii) stimulating cell proliferation; and iii) enhancing cell attachment, cell aggregation and/or cell survival.

The term “pancreatic cell” refers to a pancreatic islet, acinar, duct cell, or any other cell that is a component of the tissue in a developing or mature pancreas. Pancreatic islet cells include alpha, beta, delta, PP, and epsilon cells.

### Physiological Roles of TWEAK

The Tumor necrosis factor (TNF) superfamily of ligands and receptors are prominent regulators of cell fate decisions including survival, proliferation, and differentiation (Ware et al., Cytokine Growth Factor Rev. 14:181-4 (2003)). They play essential roles in the organogenesis and homeostasis of multiple systems, including bone, skin appendages such as hair and teeth, and lymphoid tissues. They also play complex immunoregulatory roles, for example in host defense, inflammatory responses, and positive and negative regulation of adaptive immunity.

TWEAK (TNF-like weak inducer of apoptosis), a member of the TNF ligand superfamily, is a type II-transmembrane protein that can be cleaved to function as a soluble cytokine and is highly expressed by inflammatory cells (Chicheportiche et al., J. Biol. Chem. 272: 32401-10 (1997)). The TWEAK receptor, TWEAK-R (also called Fn14), is a TNF receptor superfamily member expressed by nonlymphoid cell types. TWEAK was first described as a weak inducer of apoptosis (Chicheportiche et al., J. Biol. Chem. 272:32401-10 (1997)), and has been found to trigger multiple cellular responses ranging from proliferation to cell death through its cognate receptor TWEAK-R. TWEAK-R expression is highly inducible (Meighan-Mantha et al., J. Biol. Chem. 274:33166-76 (1999); Feng et al., Am. J. Pathol. 156:1253-61 (2000)). A comprehensive survey of TWEAK and TWEAK-R expression in normal versus injured and diseased tissues in both mice and humans recently demonstrated that in both species, expression of TWEAK, and in particular TWEAK-R, is relatively low in normal tissues but undergoes dramatic upregulation in settings of tissue injury and diseases. The studies also showed that TWEAK-R is expressed by many tissue-resident progenitor cells (Girgenrath et al., EMBO J. 25:5826-39 (2006)); Jakubowski et al., J. Clin. Invest, 115:2330-40 (2005); Perper et al., J. Immunol. 177:2610-20 (2006)).

After acute injury, inflammatory cytokines induce the influx of scavenger cells to remove dying cells and tissue debris, and promote wound closure and healing through controlled proliferation, migration of cells, and extracellular matrix turnover. More recently, the concept that inflammatory cells also regulate tissue regeneration via their effects on tissue progenitor cells has been suggested (Duffield et al., Clin. Sci. (Lond) 104:27-38 (2003)). However, in settings of chronic disease, these multifaceted activities are often dysregulated and pathogenic. It is now evident that TWEAK is a multifunctional cytokine, similar in this regard to its sibling TNF. TWEAK functions physiologically after acute injury and pathologically in chronic inflammatory disease settings. In contrast to TNF, TWEAK plays no apparent role in development or homeostasis.

New evidence has recently emerged that TWEAK can potently regulate the cell fate decisions of certain progenitor cell types which express TWEAK-R. It appears to act as a growth factor selective for liver progenitor cells (Jakubowski et al., J. Clin. Invest. 115: 2330-40 (2005)). TWEAK can upregulate pro-survival and cell cycle-related genes in progenitors of the mesenchymal lineage (Girgenrath et al., EMBO J. 25: 5826-39 (2006)), and it inhibits myotube formation and expression of muscle-specific transcription factors in myoblasts cultured under differentiation conditions (Girgenrath et al., EMBO J. 25: 5826-39 (2006); Dogra et al., J. Biol. Chem. 281: 10327-36 (2006)). Differentiation blockade by TWEAK has been recapitulated in other cell systems in vitro, including 3T3L1 adipogenesis Burkly et al, Cytokine 40:1-16 (2007); Tiller et al, Endocrinology 150:5373-5383 (2009); Alexaki et al, J. Immunol. 183:5948-5956 (2009)), chondrocyte differentiation from human mesenchymal stem cells, osteoblastogenesis from human primary osteoblast precursor cells (Perper et al., J. Immunol. 177: 2610-20 (2006); Vincent et al, J. Bone Min Res 24:1434-1449 (2009)), and mouse osteoblastic MC3T3-E1 cell terminal differentiation (Ando et al., Arthritis Res. Ther. 8: R146 (2006)). However, other studies have indicated that in some cells, TWEAK may have the opposite effect. For example, TWEAK has been shown to induce rather than inhibit osteoclast differentiation from the human monocytic cell line THP-1 (Polek et al., J. Biol. Chem. 278:32317-23 (2003)). TWEAK has also been shown to promote osteoclastogenesis indirectly by upregulating RANKL on osteoblasts (Ando et al., Arthritis Res. Ther. 8:R146 (2006)). For neuronal progenitors, TWEAK has been shown to have differing effects depending on the stage of development: TWEAK promotes neurite outgrowth in embryonic-stage progenitors without affecting their proliferation, but it decreases post-natal progenitor proliferation without having an effect on neurite outgrowth (Hamill et al., J. Neurosci. Res. (2007)). For erythroid precursor cells, TWEAK appears to inhibit growth as well as differentiation (Felli et al., J. Immunol. 175:1464-72 (2005)). Thus, the ability of TWEAK to induce progenitor cell proliferation and inhibit differentiation may vary in different tissues and stages of development.

### Expansion of Pancreatic Cells

In some embodiments, the methods of the invention comprise contacting a first population of cells comprising one or more pancreatic cells with a TWEAK-R agonist to thereby obtain a second population of cells. The second population of cells can comprise a higher number of pancreatic cells, e.g., at least about 50% more, 2-fold more, 5-fold more, 10-fold more, 25-fold more, 50-fold more, 100-fold more, or over 100-fold more than the number in the first population of cells. The second population of cells can comprise a higher number of pancreatic progenitor cells, e.g., cells having the ability to differentiate into a cell of a committed pancreatic cell type. The second population can also comprise a higher number of differentiated pancreatic cells, e.g., islet, acinar or ductal cells. For example, the second population of cells comprises a higher number of beta cells, e.g., insulin secreting cells, relative to the first population of cells. The first population of cells can be in vitro or in vivo. The first population of cells can be treated or contacted with a TWEAK-R agonist in vitro and then administered to a subject. The first population of cells can be obtained from a subject. In one embodiment, the first population of cells is obtained from a subject, contacted in vitro or ex vivo with a TWEAK-R agonist, and administered to a subject, who is the same or different from the subject from whom the first population of cells was obtained.

A first population of cells may consist of one or more cells. For example, a first population of cells may consist of at least about 10, 10², 10³, 10⁴, 10⁵, 10⁶, 10⁷ or 10⁸ cells. A first population of cells may comprise from about 1 to about 10² cells; from about 10 to about 10³ cells; from about 10² to 10⁴ cells; from about 10³ to 10⁵ cells; from about 10⁴ to 10⁶ cells; from about 10⁵ to 10⁷ cells or from about 10⁶ to 10⁸ cells. The first population of cells may consist of essentially one type of cell or of several types of cells. The first population of cells may be enriched in pancreatic cells, e.g., pancreatic duct epithelial cells. In certain embodiments, at least about 50%, 60%, 70%, 80%, 90%, 95%, 98% or 99% of the cells in the first population of cells are pancreatic cells, e.g., pancreatic ductal epithelial cells. In certain embodiments, the first population of cells does not comprise, or is essentially devoid of, cells which, if present in the first population of cells, would dominate in the second population of cells. A population of cells that is “essentially devoid” of certain cells refers to a population of cells comprising less than about 0.1%, 1%, 5%, 10%, 20%, 30%, 40% or 50% of those undesirable cells. For example, in one embodiment, the first population of cells is substantially devoid of mesenchymal cells and/or connective tissue cells.

A first population of cells may be incubated with a TWEAK-R agonist for a time sufficient for the desirable number and type of pancreatic cells to be expanded. For example, a first population of cells may be incubated for about 5, 10, 18, 24, 30, 36, 42, or 48 hours with a TWEAK-R agonist.

In certain embodiments, a first population of cells is treated with a TWEAK-R agonist in vitro and administered to a subject. A first population of cells may also be administered to a subject and the subject is treated with a TWEAK-R agonist. In certain embodiments, a first population of cells is treated with a TWEAK-R agonist in vitro, the population is administered to a subject, and the subject is treated with a TWEAK-R agonist. When a TWEAK-R agonist is administered to a subject, it is administered in an amount and for a time sufficient for the desirable population of pancreatic cells to be regenerated or expanded.

In exemplary embodiments, the methods comprise administering to a subject a therapeutically effective amount of a TWEAK receptor agonist to induce the proliferation or appearance of pancreatic progenitor cells in the pancreas of the subject. The term “pancreatic progenitor cell” refers to a cell having the ability to differentiate into a cell of a committed pancreatic cell type (or lineage). In further embodiments, the methods include inducing expansion of pancreatic progenitor cells in vitro by treating a culture of isolated pancreatic cells with an amount of a TWEAK-R agonist effective for enhancing the survival, surface-adherence and/or proliferation of the pancreatic progenitor cells.

In some embodiments, pancreatic progenitor cells are expanded ex vivo using a TWEAK-R agonist to induce the proliferation of the cells in culture. Pancreatic progenitor cells expanded in culture may be transplanted into a subject in need thereof. The pancreatic progenitor cells to be expanded in vitro may be pancreatic progenitor cells isolated from pancreatic tissue surgically obtained from the individual into which they are later transplanted. Other cells to be expanded in vitro may be pancreatic progenitor cells derived from cadaveric pancreatic tissue, adult stem cells, embryonic stem cells (ESCs), epiblast stem cells (EpiSCs), totipotent stem cells, and/or induced pluripotent stem cells (iPSCs; somatic cells that have been reprogrammed to a pluripotent state). Exemplary iPSCs are stem cells of adult origin into which the genes Oct-4, Sox-2, c-Myc, and Klf have been transduced, as described by Takahashi and Yamanaka (Cell 126(4):663-76 (2006)). Other exemplary iPSC's are adult stem cells into which OCT4, SOX2, NANOG, and LIN28 have been transduced (Yu, et al., Science 318:1917-1920 (2007)). Populations of pancreatic cells to be expanded in culture can include CK-, Ki-67, -Pdx1-, and/or Ngn3-positive cells. Progenitor cells isolated from tissue surgically obtained from an individual can be transplanted back into the same individual after expansion in vitro using a TWEAK-R agonist according to the methods of the invention. In some embodiments, expansion of pancreatic cells involves proliferation of the cells. “Expansion” of a cell population may also include additional events, e.g., promotion of attachment, aggregation and/or survival, of the population of cells or a subpopulation thereof. The term “expansion” refers to expanding the population of cells in a culture, including, for example, expanding the cell population by at least 10%, 30%, 50%, 75%, 2-fold, 3-fold, 4-fold, 5-fold, or 10-fold. For example, in one embodiment, more pancreatic cells are recovered from a population of cells treated with a TWEAK-R agonist relative to a similar or equivalent population of cells (e.g., same proportion of cell types) that was not treated with the TWEAK-R agonist.

The pancreatic cells to be expanded in vitro can be isolated from surgically-obtained pancreatic tissue samples or donated organ tissue and cultured as described by Yatoh et al. (Diabetes 56:1802-9 (2007)), using, for example, the standardized methodology of Linetsky et al. (Diabetes 46:1120-23 (1997)) or similar methods. As described by Yatoh et al., following islet isolation and purification, cells can be further purified by enzymatic dispersion of the isolated cells, such as by exposure to trypsin, and optionally filtering, such as with a 40-μm cell strainer, followed by immunoaffinity sorting using microbeads, immunomagnetic beads, or other similar immunoaffinity bulk sorting tools. An antibody recognizing a pancreas-specific cell marker can be used in the immunoaffinity sorting, such as an antibody directed to CA19-9, a cell-surface marker found throughout the pancreatic ductal tree (Lefebvre et al., Diabetes 47:134-137 (1998); Bouwens et al., Diabetologia 41:629-633 (1998); Gmyr et al., Biochem Biophys Res. Com. 320:27-33 (2004)) or to Prominin I (CD133; Hori et al., Stem Cells 26(11):2912-20 (2008)). Crude or purified cells can be cultured in non-tissue culture-treated cell culture flasks in CMRL medium (for example Invitrogen Gibco™ CMRL Medium-1066) at, for example, 10⁶ cells per 25 cm² of flask surface area or otherwise appropriate cell density. A TWEAK-R agonist is added to the growth media of the cultured cells. The TWEAK-R agonist mayn be present at a concentration that enhances the survival, adherence and/or proliferation of the pancreatic cells relative to that of cells not provided the TWEAK-R agonist. Exemplary concentrations of a TWEAK-R agonist can be in the range of, for example, 1 to 100 pg/ml or less, 100 to 1000 pg/ml, 1 to 100 ng/ml, or 100 to 1000 ng/ml or more. In some embodiments, the concentration of a TWEAK-R agonist can be between 1 and 100 ng/ml, including, for example, 1, 10, 20, 30, 40, 50, 60, 70, 80, 90, and 100 ng/ml.

### Diabetes and Other Medical Uses

In some embodiments, the methods are useful for the treatment of diabetes. In exemplary embodiments, subjects may be treated with an amount of a TWEAK-R agonist that is sufficient to induce regeneration of insulin-secreting pancreas cells by increasing the numbers of pancreatic progenitor cells (i.e., a therapeutically effective amount) in the subject. In some embodiments, the subject is diagnosed with diabetes or a related condition and is then administered a TWEAK-R agonist. Diabetes-related conditions include, but are not limited to, syndrome x (metabolic syndrome), insulin resistance, nephropathy, ketoacidosis, hyperosmolar hyperglycemic nonketoic syndrome, gastroparesis, diabetic kidney disease, and other diabetes related conditions. In some embodiments, the method of treating diabetes comprises administering an amount of a TWEAK-R agonist effective for inducing the regeneration of insulin-secreting pancreas cells from transplanted progenitor cells to a subject with diabetes who has received a transplant of progenitor cells capable of giving rise to insulin-secreting pancreas cells. In some embodiments, the subject has received a transplantation of cadaveric or surgically obtained cells containing pancreatic progenitor cells or cells capable of dedifferentiating into pancreatic progenitor cells, wherein subsequent administration of a TWEAK-R agonist induces the regeneration of pancreatic progenitor cells in the transplanted islet tissue. In further embodiments, the subject has received a stem cell transplant in the pancreas, wherein subsequent administration of a TWEAK-R agonist induces the regeneration of the transplanted stem cells. In some embodiments, the pancreatic stem cell transplant and the TWEAK-R agonist are administered simultaneously. In these and further embodiments, a variety of potential sources of pancreatic progenitor cells, as detailed above, may be transplanted, followed by administration of a TWEAK-R agonist to promote the regeneration of pancreatic tissue.

One embodiment of the invention includes a method of treating diabetes comprising a) culturing progenitor cells in vitro, wherein the progenitor cells are pancreatic progenitor cells isolated from a subject with diabetes or a living or cadaveric donor, adult stem cells from said subject, embryonic stem cells, or induced pluripotent stem cells, b) treating with an effective amount of a TWEAK-R agonist to generate an expanded population of the progenitor cells; and c) transplanting the expanded population of progenitor cells into the pancreas of the subject with diabetes, wherein insulin-producing pancreatic cells are regenerated in the subject from the transplanted progenitor cells. In some embodiments, the method further comprises administering, after the transplantation, an effective amount of a TWEAK-R agonist to promote survival, expansion, and/or migration in vivo of the transplanted cells and cells derived therefrom.

In some embodiments, a TWEAK-R agonist is administered to induce proliferation/and or appearance of pancreatic progenitor cells in a subject who has type 1 diabetes (insulin dependent diabetes mellitus; IDDM; T1 D; juvenile diabetes), who has lost functional beta cells as a result of the pathological autoimmune response targeting these cells. In other embodiments, a TWEAK-R agonist is administered to induce proliferation and/or appearance of pancreatic progenitor cells in a subject with type 2 diabetes. Type 2 diabetes (non-insulin dependent diabetes mellitus; NIDDM; T2D; adult-onset diabetes) is a complex metabolic disease that involves lowered sensitivity to insulin, and can be associated with decreased functional mass of beta cells in the pancreas. In some embodiments, administration of a TWEAK-R agonist to a type 2 diabetic is combined with therapy for improved insulin sensitivity. In some embodiments, the subject has maturity onset diabetes of the young (MODY).

In some embodiments, the subject administered a TWEAK-R agonist for the induction of pancreatic progenitor cell proliferation has an inflammatory condition. Exemplary inflammatory conditions include, but are not limited to, rheumatoid arthritis, asthma, inflammatory bowel disease, vasculitis, transplant rejection, reperfusion injury, pelvic inflammatory disease, glomerulonephritis, chronic prostatitis, chronic obstructive pulmonary disease, psoriasis, atherosclerosis, and osteoarthritis. Accordingly, in some embodiments, the TWEAK-R agonist may be co-administered with an anti-inflammatory agent. Anti-inflammatory agents can include immunosuppressants, TNF inhibitors, corticosteroids, non-steroidal anti-inflammatory drugs (NSAIDs), disease-modifying anti-rheumatic drugs (DMARDS), and the like. Exemplary anti-inflammatory agents include, for example, prednisone; methylprenisolone (Medrol®), triamcinolone, methotrexate (Rheumatrex®, Trexall®), hydroxychloroquine (Plaquenil®), sulfasalzine (Azulfidine®), leflunomide (Arava®), etanercept (Enbrel®), infliximab (Remicade®), adalimumab (Humira®), rituximab (Rituxan®), abatacept (Orencia®), interleukin-1, anakinra (Kineret™) ibuprofen, ketoprofen, fenoprofen, naproxen, aspirin, acetominophen, indomethacin, sulindac, meloxicam, piroxicam, tenoxicam, lornoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine or sulfasalazine and the like.

In some embodiments, the subject to be administered a TWEAK-R agonist does not have an inflammatory condition.

In some embodiments, regeneration of pancreatic tissue can be induced using an effective amount of a TWEAK-R agonist in a subject in need of regeneration of pancreatic cells due to illness, traumatic injury, or chemical treatment that has damaged pancreatic tissue. In some embodiments, the subject has undergone a pancreatectomy as a result of illness or traumatic injury. In some embodiments, the illness is pancreatic cancer. Pancreatic cancers include adenocarcinomas, serous cystadenomas, acinar cell cancers, and pancreatic neuroendocrine tumors such as insulinomas. In some embodiments, the subject is a patient with chronic pancreatitis who has undergone a pancreatectomy or has lost functional pancreatic tissue as a result of the chronic inflammation of the pancreas associated with chronic pancreatitis. In some embodiments, the subject has chronic pancreatitis as a result of a hereditary disorder of the pancreas, extended and heavy alcohol consumption, cystic fibrosis, hypercalcemia (high levels of calcium in the blood), hyperlipidemia, hypertriglyceridemia (high levels of blood fats), a reaction to certain medicines, an autoimmune condition, or an unknown cause. Cystic fibrosis is the most common inherited pancreatic disease, ultimately resulting in the pancreas becoming badly scarred and shrinking. Accordingly, one embodiment includes treating pancreatic disorders associated with cystic fibrosis. In some embodiments, a TWEAK-R agonist is administered to induce pancreatic cell regeneration in a subject who has undergone a pancreatectomy, including but not limited to subjects with pancreatic cancer, wherein the pancreatic cells regenerate from transplanted pancreatic progenitor cells or stem cells. Subjects in need of pancreatic regeneration also include subjects having lost one or more cells or one or more functions of the pancreas, including, for example, a subject having a pre-diabetic condition, insulin resistance, impaired glucose tolerance, or the like.

Regeneration of insulin-producing pancreatic cells in a subject can be measured by detecting increased serum levels of C-peptide, transmembrane protein 27 (Tmem27), decreases in glycosylated hemoglobin (Hb_{A1c}), and the like, or by detecting a reduced requirement for insulin in maintaining normal blood glucose levels. C-peptide is a peptide which is released when proinsulin is proteolytically cleaved to form insulin prior to insulin secretion from beta cells. Tmem27, also known as collectrin, is a cell-surface, glycosylated protein that is cleaved and shed from the plasma membrane of beta cells. This cleavage process is beta cell specific and does not appear to occur in other cell types (Akpinar, et al., Cell Metab. 2(6):385-97 (2005)). Hb_{Aic} is a glycosylated form of hemoglobin used primarily to identify the average plasma glucose concentration over prolonged periods of time. It is formed in a non-enzymatic pathway by hemoglobin's normal exposure to high plasma levels of glucose.

In some embodiments, the TWEAK-R agonist is administered in or near the pancreas or pancreatic region. The term “pancreatic region” refers to the region where the pancreas would otherwise reside for those patients who have lost some or most of their pancreas. In some embodiments, the TWEAK-R agonist is administered in and/or near the liver.

In exemplary embodiments, the pancreatic progenitor cells are duct epithelial cells. In further embodiments, the progenitor cells are cells in a ductal adjacent region.

In some embodiments, the subject administered a TWEAK-R agonist for activation of endogenous or transplanted progenitor cells is a mammal. In exemplary embodiments, the subject is a human. In aalternate embodiments, the subject is a rodent, such as, e.g., a mouse or a rat.

### TWEAK-R Agonists

The terms “TWEAK receptor (TWEAK-R) agonist” and “TWEAK-R activating agent” refer to any agent which can augment signaling of the TWEAK receptor (Fn14), or that can influence how the receptor signal is interpreted within the cell. The TWEAK receptor protein, Fn14, is characterized in International Application No. PCT/US2001/028451), which is incorporated herein by reference in its entirety. The human and mouse amino acid sequences for this type I transmembrane protein are provided herein as SEQ ID NO:3 and SEQ ID NO:4, respectively. Exemplary TWEAK-R agonists include TWEAK, a TWEAK fusion protein such as Fc-TWEAK, a TWEAK analog, and a soluble anti-TWEAK-R agonistic antibody. The term “signaling of the TWEAK-R” refers to all molecular reactions associated with the TWEAK-R pathway and subsequent molecular reactions which result therefrom.

TWEAK as a TWEAK-R Agonist

In exemplary embodiments, the TWEAK-R agonist is TWEAK. In some embodiments, TWEAK is human TWEAK (SEQ ID NO:1). In alternate embodiments, TWEAK is mouse TWEAK (SEQ ID NO:2).

In some embodiments, TWEAK is membrane bound, and can be delivered in pharmaceutical compositions that comprise liposomes or other cellular or pseudocellular delivery systems. In some embodiments, the TWEAK polypeptide comprises a portion of SEQ ID NO:1 or SEQ ID NO:2 wherein the polypeptide is soluble TWEAK. Proteins in the TNF family of ligands are characterized by a short N-terminal stretch of normally short hydrophilic amino acids, often containing several lysine or arginine residues thought to serve as stop transfer sequences. This N-terminal region is followed by a transmembrane segment and then an extracellular region of variable length that separates the C-terminal receptor binding domain from the membrane. This region is sometimes referred to as the “stalk”. Though TWEAK is synthesized as a type-II transmembrane protein in the endoplasmic reticulum, TWEAK is also secreted as a soluble cytokine following proteolytic cleavage within its stalk region by members of the furin protease family while it traverses the trans Golgi network (Chicheportiche, et al., J. Biol. Chem. 272:32401-10 (1997)).

Soluble TWEAK polypeptides can include all or part of the stalk sequence as long as the polypeptide is secreted from the cell in which it is produced. Thus, to create a soluble secreted form of TWEAK, one would remove at the DNA level the N-terminal transmembrane regions and some portion of the stalk region, and optionally replace them with a leader sequence containing a proteolytic cleavage site, such as a Tev protease cleavage site, appropriate for use in the chosen expression system. A skilled artisan could vary the amount of the stalk region retained in the secretion expression construct to optimize both receptor binding properties and secretion efficiency. For example, constructs containing all possible stalk lengths, i.e. N-terminal truncations, can be prepared such that polypeptides starting at a position including and between amino acids 46 and 104 of human TWEAK (SEQ ID NO:1) would result. In some embodiments, the soluble TWEAK polypeptide comprises or consists of amino acids 46 to 249 of SEQ ID NO:1. In other embodiments, the polypeptide comprises or consists of amino acids 104 to 249 of SEQ ID NO:1. In other exemplary embodiments, the soluble TWEAK polypeptide has an amino terminus at any position from amino acid 46 to 104 of SEQ ID NO:1 and a carboxy terminus at position 249 of SEQ ID NO:1. In further embodiments, TWEAK is a soluble mouse TWEAK polypeptide having an amino terminus at any position including and between amino acids 46 and 104 of SEQ ID NO:2 and a carboxy terminus at position 249 of SEQ ID NO:2. Soluble forms of TWEAK can signal effectively and hence can be administered as a drug which mimics the naturally secreted form and the extracellular domain of the membrane-anchored form. TWEAK polypeptides and soluble versions thereof useful in the invention are described in U.S. Pat. No. 7,109,298, which is incorporated herein by reference in its entirety.

A DNA sequence encoding a desired soluble polypeptide may be subcloned into an expression vector for production of the polypeptide, or the desired encoding DNA fragment may be chemically synthesized. Purification of the polypeptides from recombinant host cells is facilitated by the fact that the polypeptides are secreted, and soluble proteins are generally suited for parenteral administration according to some embodiments of the invention. A secreted soluble polypeptide may be identified (and distinguished from its non-soluble membrane-bound counterparts) by separating intact cells which express the desired polypeptide from the culture medium, e.g., by centrifugation, and assaying the medium (supernatant) for the presence of the desired polypeptide.

TWEAK Analogs

The term “TWEAK analog” refers to a polypeptide that is derived from a native TWEAK polypeptide but differs in its amino acid sequence. TWEAK polypeptides with changes in their amino acid sequence may be mutated forms of TWEAK, TWEAK fusion proteins, and TWEAK fragments. A TWEAK analog possesses TWEAK-R agonist activity.

In some embodiments, TWEAK analog is a mutated form of TWEAK containing 1 to 5, 5 to 10, 10 to 15, 15 to 20, 20 to 25, 25 to 30, 30 to 35, or 35 to 40 different amino acids when compared to the wild-type sequence. This type of TWEAK analog will have an amino acid sequence that is at least 80%, 85%, 90%, 95%, 97%, or 99% identical to native TWEAK, such as, e.g., the polypeptide of SEQ ID NO:1, amino acids 46 to 249 of SEQ ID NO:1 and amino acids 104 to 249 of SEQ ID NO:1. In specific embodiments, the TWEAK analog is a polypeptide that is at least 80% identical to SEQ ID NO:1 or SEQ ID NO:2. In other specific embodiments, the TWEAK analog is a polypeptide that is at least 85% identical to SEQ ID NO:1 or SEQ ID NO:2. In other embodiments, the TWEAK analog is a polypeptide that is at least 90% or at least 95% identical to SEQ ID NO:1 or at least 90% or at least 95% identical to SEQ ID NO:2.

Generally, conservative substitutions of one or more amino acids present in the native TWEAK polypeptide can be made without adversely effecting the activity of the polypeptide. Examples of conservative substitutions include substitution of amino acids outside of regions of TWEAK that are conserved between species (such as between human and mouse TWEAK), and substitution of amino acids that do not alter the secondary and/or tertiary structure of TWEAK. Specific examples include substitution of one aliphatic residue for another, such as Ile, Val, Leu, or Ala for one another, or substitution of one polar residue for another, such as between Lys and Arg; Glu and Asp; or Gln and Asn, or substitution of one aromatic residue for another, such as Phe, Trp, or Tyr for one another. Other conservative substitutions, such as substitutions of entire regions having similar hydrophobicity characteristics, are known in the art and are contemplated in the methods of the invention. Methods of generating mutated forms of TWEAK are well known in the art of molecular biology, and include altering DNA molecules by random mutagenesis, site directed mutagenesis, deletions, and truncations. Specific techniques include polymerase chain reaction (PCR) mutagenesis, saturation (i.e. chemical or radiation) mutagenesis, chemical DNA synthesis, alanine scanning mutagenesis, oligonucleotide-mediated mutagenesis (hybridization to a DNA template in vitro followed by enzymatic elongation), cassette (recombinant) mutagenesis, and combinatorial mutagenesis (introduction of random degenerate sequences into the TWEAK DNA).

In certain exemplary embodiments, the TWEAK analog is a TWEAK fusion protein. The term “fusion protein” refers to a chimeric protein comprising amino acid sequences of two or more different proteins. In some embodiments, the TWEAK fusion protein includes, in addition to TWEAK, one or more polypeptide portions that enhance one or more of in vivo stability, in vivo half-life, uptake/administration, tissue localization or distribution, formation of protein complexes, and/or purification. Fusion proteins may be generated recombinantly using molecular cloning techniques well known in the art. In some embodiments, the fusion protein contains a TWEAK polypeptide as described above, including a polypeptide comprising or consisting of the sequence of SEQ ID NO:1, a polypeptide with the sequence of SEQ ID NO:2, or soluble forms thereof as described herein. In certain embodiments, the TWEAK fusion protein may contain a mutated form of TWEAK as described above. In other exemplary embodiments, the TWEAK fusion protein includes a purification subsequence, such as an epitope tag, a FLAG tag, a polyhistidine sequence, or GST polypeptide.

In certain exemplary embodiments, the TWEAK-R agonist is a TWEAK fusion protein that includes the Fc domain of an immunoglobulin such as, e.g., IgG1, IgG2, IgG3, IgG4), IgE, IgD, IgM (“Fc-TWEAK”). As used herein, the Fc portion of an immunoglobulin has the meaning commonly given to the term in the field of immunology. Specifically, this term refers to an antibody fragment which does not contain the two antigen binding regions (the Fab fragments) from the antibody. The Fc portion consists of the constant region of an antibody from both heavy chains, which associate through non-covalent interactions and disulfide bonds. The Fc portion can include the hinge regions and extend through the CH₂ and CH3 domains to the C-terminus of the antibody. The Fc portion can further include one or more glycosylation sites. In some embodiments, the immunoglobulin Fc portion of the fusion protein contains mutations designed to remove unwanted effector functions and/or reduce the risk of inducing an immune response after repeated and prolonged administration, as described in U.S. Pat. No. 7,452,966. The fusion proteins can comprise, for example, glutathione-S transferase (GST), green fluorescent protein (GFP), maltose binding protein (MBP), a 6×His tag, a Flag tag, and the like, and/or can be conjugated to polyethylene glycol (e.g., PEGylated), e.g., to reduce the immunogenicity and/or increase the circulating half-lives of the fusion protein.

TWEAK Mimetics

In some embodiments, the TWEAK-R agonist is a TWEAK mimetic or TWEAK peptide mimetic. The term “TWEAK mimetic” or “peptide mimetic” refers to a non-peptide analog of the type commonly used in the pharmaceutical industry as drugs with properties analogous to those of the template peptide (Fauchere, J. Adv. Drug Res. 15: 29 (1986); Veber and Freidinger, TINS p. 392 (1985); and Evans et al., J. Med. Chem. 30: 1229 (1987), incorporated herein by reference). Mimetics are often developed with the aid of computerized molecular modeling. Peptide mimetics that are structurally similar to therapeutically useful peptides may be used to produce an equivalent therapeutic or prophylactic effect. Generally, mimetics are structurally similar to a paradigm polypeptide (for example, a polypeptide that has a desired biochemical property or pharmacological activity), such as a TWEAK fragment, but have one or more peptide linkages optionally replaced by a linkage selected from the group consisting of: —CH₂NH—, —CH₂S—, —CH₂—CH₂—, CH═CH—(cis and trans), —COCH₂—, —CH(OH)CH₂—, and —CH₂SO—, by methods well known in the art. Systematic substitution of one or more amino acids of a consensus sequence with a D-amino acid of the same type (for example, D-lysine in place of L-lysine) may also be used to generate more stable peptides. In addition, constrained peptides comprising a consensus sequence or a substantially identical consensus sequence variation may be generated by methods known in the art (Rizo and Gierasch, Ann. Rev. Biochem. 61: 387 (1992), incorporated herein by reference), for example, by adding internal cysteine residues capable of forming intramolecular disulfide bridges which cyclize the peptide. TWEAK analogs can differ in sequence from the naturally occurring TWEAK ligand amino acid sequence or can differ in ways that do not involve the sequence, as described herein, or both.

Agonistic Anti-TWEAK-R Antibodies

Like other members of the TNF ligand and TNF ligand receptor families, the binding complex of TWEAK and TWEAK-R consists of a TWEAK homotrimer symmetrically bound to three TWEAK-R monomers. Historically, TNF receptor family members have been thought to be activated by ligand-induced trimerization of receptor monomers. However, recent evidence has indicated that TNF receptors may exist as pre-assembled oligomers on the cell surface, and that ligand-induced signaling is transmitted by the preformed receptor oligomers. (Reviewed in Chan, Cytokine. 37(2):101-7 (2007)). Although antibodies directed to TNF family receptors can act as antagonists that block receptor-ligand interaction, they can also act as agonists that induce oligomerization, receptor clustering, and/or otherwise activate receptor signaling (Pukac, et al., Br. J. Cancer. 92:1430-1441 (2005); Desbarats and Newell, Nat. Med. 6(8):920-3 (2000)). Antibodies specific for TWEAK-R that bind and induce the receptor's activity have been described (see, e.g., U.S. Pat. No. 7,208,151, incorporated herein by reference in its entirety, and International application No. PCT/EP2006/004974). Further exemplary antibodies are described in US Patent publication number 20090324602, which is incorporated by reference herein in its entirety. The heavy and light chain sequences of an exemplary antibody specific for TWEAK-R that induces the receptor's activity are provided in SEQ ID NO:5 and 6, respectively.

TWEAK-R antibodies that induce TWEAK-R activity by binding to and potentiating TWEAK-R oligomerization/clustering or by otherwise activating TWEAK-R are specifically contemplated in the methods of the invention. In some embodiments, the methods include the use of single anti-TWEAK-R antibodies, including single anti-TWEAK-R monoclonal antibodies. In other embodiments, the methods include the use of multiple anti-TWEAK-R antibodies in solution which act as TWEAK-R agonists. Polyclonal anti-TWEAK-R antibodies directed against different epitopes of TWEAK-R can be used. Multiple anti-TWEAK-R monoclonal antibodies directed against different and non-overlapping epitopes of TWEAK-R can also be used.

The terms“anti-TWEAK-R monoclonal antibody” refers to any monoclonal antibody that recognizes and binds to a single epitope of TWEAK-R. The use of anti-TWEAK-R monoclonal antibodies as TWEAK-R cross-linking agents relies on the use of monoclonal antibodies recognizing one epitope or two or more non-overlapping epitopes. Additional epitopes (as defined by new monoclonal antibodies) may be identified by continuing to generate new hybridomas from the spleen cells of mice immunized with TWEAK-R or fragments thereof, by immunizing different species of animals, and by using different routes of immunization.

Epitopes can also be directly mapped by assessing the ability of different monoclonal antibodies to compete with each other for binding to TWEAK-R using surface plasmon resonance-coupled chromatographic techniques (Pharmacia BlAtechnology Handbook, “Epitope Mapping”, Section 6.3.2, (May 1994); see also Johne et al., J. Immunol. Methods, 160(2): 191-8 (1993)).

Anti-TWEAK-R IgM Monoclonal Antibodies as TWEAK-R Agonists

Anti-TWEAK-R monoclonal antibodies which comprise more than the usual two IgG antigen binding sites will also function in solution as multi-valent, cell-surface TWEAK-R cross-linking agents, and will accordingly fall within the definition of a TWEAK-R agonist according to this invention. The term “multivalent ligand” refers to a molecule or complex which has more than one receptor binding site and which is capable of simultaneously binding and bringing into close proximity at least two receptor molecules. For example, the antigen binding sites of an anti-TWEAK-R monoclonal antibody can be built into IgM molecules (which have ten antigen binding sites) using standard recombinant DNA and hybridoma techniques.

Alternatively, one may collect and enrich for complete mouse (or other animal) IgM molecules isolated by hybridoma fusion techniques after a single immunization with antigen. One way to enrich for IgM molecules is to immunize CD40 signaling-deficient mice (Kawabe et al., Immunity, 1: 167-78 (1994); Xu et al., Immunity, 1: 423-31 (1994)). These mice cannot effectively produce IgGs and therefore their response to challenge by antigen is enriched for IgM isotypes.

Anti-TWEAK-R IgM antibodies, by virtue of their increased valency, can effectively aggregate TWEAK-R molecules within the plane of the membrane, thereby enhancing TWEAK-R signaling as compared to their IgG counterparts having two antigen binding sites. A dramatic example of the increased efficiency of multivalent antibodies in receptor clustering is seen with antibodies to the Fas receptor, where the IgM form is very potent and normal bivalent IgGs are not effective in solution (Yonihara and Yonihara, J. Exp. Med., 169:1747-56 (1989); Alderson et al., Int. Immunol., 6: 1799-1806 (1994)).

Likewise, the apo-1 monoclonal antibody to the Fas receptor is an IgG3 monoclonal antibody. This monoclonal antibody potently activates the Fas receptor, relying on Fc interactions unique to IgG3 subtypes to aggregate into larger polyvalent forms. Removal of the Fc region creates a F(ab)₂ form that cannot associate into larger aggregates and which is inactive (Dhein et al., J. Immunol., 149: 3166 73 (1992)). Thus by analogy, it is predicted that IgM versions of anti-TWEAK-R monoclonal antibodies will be potent activators of TWEAK-R.

In some embodiments, the TWEAK-R agonist is a complex of cross-linked anti-TWEAK-R monoclonal antibodies. The term “cross-linked anti-TWEAK-R monoclonal antibodies” refers to antibodies directed against TWEAK-R which have either been intermolecularly cross-linked to each other to form antibody agglomerates in solution using an anti-TWEAK-R antibody or monoclonal antibody cross-linking agent, or which have been immobilized in close proximity to one another on a surface or matrix such as a microbead. The term “anti-TWEAK-R antibody (or monoclonal antibody) cross-linking agent” refers to any agent which can covalently or non-covalently aggregate anti-TWEAK-R antibodies in solution so that the antibodies can bind to and potentiate target cell surface TWEAK-R signaling. Such cross-linking agents include but are not limited to chemical cross-linking agents, secondary antibodies which react with portions of the anti-TWEAK-R antibodies or monoclonal antibodies, and soluble or surface bound Fc receptors (either endogenous or added exogenously) which can bind to anti-TWEAK-R antibodies.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, including, for example, intact and full length immunoglobulins of types IgA, IgG (including, for example, IgG1, IgG2, IgG3, IgG4), IgE, IgD, IgM (as well as subtypes thereof). The term also encompasses bispecific antibodies, bispecific antibody fragments, multimeric antibodies, and multimeric antibody fragments. The light chains of the immunoglobulin may be of types kappa or lambda. In some embodiments, the antibody is glycosylated.

The VH and VL regions can be further subdivided into regions of hypervariability, termed “complementarity determining regions” (“CDR”), interspersed with regions that are more conserved, termed “framework regions” (FR). The extent of the FR's and CDRs has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al., J. Mol. Biol., 196: 901-917 (1987)). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain.

In some embodiments, one or more regions of the agonistic antibody can be human, effectively human, or humanized. An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human. A “humanized” immunoglobulin variable region is an immunoglobulin variable region that is modified such that the modified form elicits less of an immune response in a human than does the non-modified form. Descriptions of “humanized” immunoglobulins include, for example, U.S. Pat. Nos. 6,407,213 and 5,693,762. For example, one or more of the variable regions can be human or effectively human. One or more of the CDRs, e.g., VH CDR1, VH CDR2, VH CDR3, VL CDR1, VL CDR2, and VL CDR3, can be human. Each of the light chain CDRs can be human. VH CDR3 can be human. One or more of the framework regions can be human, for example, FR1, FR2, FR3, and FR4 of the heavy chain or light chain. In some embodiments, all the framework regions are human. In one embodiment, the human sequences are germline sequences (sequences encoded by a germline nucleic acid). One or more of the constant regions can be human, effectively human, or humanized. In other embodiments, at least 70, 75, 80, 85, 90, 92, 95, or 98% of the framework regions (including FR1, FR2, and FR3, collectively, or FR1, FR2, FR3, and FR4, collectively) or the entire antibody can be human, effectively human, or humanized. In some cases, humanized immunoglobulins can include a non-human amino acid at one or more framework amino acid positions. For example, FR1, FR2, and FR3 collectively can be at least 70, 75, 80, 85, 90, 92, 95, 98, or 99% identical, or completely identical, to a human sequence encoded by a human germline segment.

The antibodies can be conjugated to a moiety, e.g., can be conjugated to poly(ethylene glycol) (e.g., PEGylated), e.g., to reduce the immunogenicity and/or increase the circulating half-lives of antibodies.

Antibody Generation

Antibodies that bind to a TWEAK-R can be generated by a variety of means, including immunization, e.g., using an animal, or in vitro methods such as phage display, according to standard protocols (see, for example, “Antibodies: A Laboratory Manual,” ed. by Harlow and Lane, Cold Spring Harbor press: 1988). All or part of a TWEAK receptor or cells expressing a TWEAK receptor can be used as an immunogen or as a target for selection. For example, a TWEAK receptor or fragment thereof or TWEAK receptor-expressing cell can be used as an immunogen. In one embodiment, the immunized animal contains immunoglobulin-producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, for example, XENOMOUSE™, Green et al., Nat. Gen., 7: 13-21 (1994); U.S. Patent Publication No. 2003/0070185; U.S. Pat. No. 5,789,650; and International Patent Publication No. WO 96/34096.

Non-human antibodies to a TWEAK receptor can also be produced, for example, in a rodent. The non-human antibody can be humanized, e.g., as described in EP 239 400; U.S. Pat. Nos. 6,602,503; 5,693,761; and 6,407,213, deimmunized, or otherwise modified to make it effectively human as described above.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for C_{H} and kappa for CO can be added and the humanized heavy and light chain genes can be co-expressed in mammalian cells to produce soluble humanized antibody. Other methods for humanizing antibodies can also be used. For example, other methods can account for the three dimensional structure of the antibody, framework positions that are in three-dimensional proximity to binding determinants, and immunogenic peptide sequences. See, for example, International Application No. WO 90/07861; U.S. Pat. Nos. 5,693,762; 5,693,761; 5,585,089; 5,530,101; and 6,407,213; and Tempest et al., Biotechnology 9: 266-271 (1991).

Fully human monoclonal antibodies that bind to a TWEAK receptor can be produced, e.g., using in vitro-primed human splenocytes, as described by Boerner et al., J. Immunol., 147:86-95 (1991). They may be prepared by repertoire cloning as described by Persson et al., Proc. Nat. Acad. Sci. U.S.A, 88: 2432-2436 (1991), or by Huang and Stollar, J. Immunol. Methods, 141: 227-236 (1991); or as described in U.S. Pat. No. 5,798,230. Large nonimmunized human phage display libraries may also be used to isolate high affinity antibodies that can be developed as human therapeutics using standard phage technology (see, for example, Hoogenboom et al., Immunotechnology, 4:1-20 (1998); Hoogenboom et al., Immunol Today, 2: 371-378 (2000); and U.S. Patent Publication No. 2003/0232333).

### Antibody and Protein Production

Antibodies and other proteins described herein can be produced in prokaryotic and eukaryotic cells. In one embodiment, the antibodies are expressed in a yeast cell such as Pichia (see, for example, Powers et al., J. Immunol. Methods, 251: 123-35 (2001)), Hanseula, or Saccharomyces.

Antibodies, particularly full length antibodies, for example, IgG's, can be produced in mammalian cells. Exemplary mammalian host cells for recombinant expression include Chinese Hamster Ovary (CHO cells) (including dhfr⁻ CHO cells, described in Urlaub and Chasin, Proc. Natl. Acad. Sci. U.S.A, 77: 4216-4220 (1980), in which recombinant constructs include a DHFR selectable marker, as described in Kaufman and Sharp, Mol. Biol., 159: 601-621 (1982)), lymphocytic cell lines including NSO myeloma cells and SP2 cells, COS cells, K562 cells, and cells from a transgenic animal, such as a transgenic mammal. For example, the cells are mammary epithelial cells.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody or antibody fragment, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fc fusions) may also include modifications, including, for example, modifications that alter Fc function. Such modifications include changes that decrease or remove interaction with an Fc receptor or with Clq, or both. For example, the human IgG1 constant region can be mutated at one or more residues, including, for example, one or more of residues 234 and 237, according to the numbering in U.S. Pat. No. 5,648,260. Other exemplary modifications include those described in U.S. Pat. No. 5,648,260.

For agonistic antibodies and some TWEAK fusion proteins that include an Fc domain, the antibody/protein production system may be designed to synthesize the fusion protein or antibody with a glycosylated Fc region. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

### Formulations and Routes of Administration

The methods of this invention include the administration of an effective dose of a TWEAK-R agonist to a subject to induce regeneration of pancreatic tissue. Determination of a preferred pharmaceutical formulation and a therapeutically efficient dose regiment for a given subject is well within the skill of the art taking into consideration, for example, the condition and weight of the patient, the extent of desired treatment and the tolerance of the patient for the treatment.

The TWEAK-R agonist can be administered by any route of administration which is compatible with the agonist, and may be formulated with any pharmaceutically acceptable carrier appropriate to the route of administration. Such carriers are well known to those skilled in the art. Administration can be performed, for example, intravenously, intraperitoneally, orally, via implant, transmucosally, transdermally, intramuscularly, and subcutaneously. Preferred routes of administration are parenteral and, in particular, intravenous, intraperitoneal, and intracapsular. Treatments can be conducted over an extended period on an outpatient basis. Daily dosages of the therapeutic agents are expected to be in the range of about 0.01 to 1000 μg/kg body weight, and more preferably about 10 to 300 μg/kg body weight, although precise dosages will vary depending upon the particular therapeutic agent employed and the particular subject's medical condition and history.

The following delivery systems, which employ a number of routinely used carriers, are only representative of the many embodiments envisioned for administering the TWEAK-R agonist according to the methods of the invention.

In some embodiments, the TWEAK-R agonist is administered via an injectable drug delivery system. Such systems can include solutions, suspensions, gels, microspheres and polymeric injectables, and can comprise excipients such as solubility-altering agents (including, for example, ethanol, propylene glycol and sucrose) and polymers (including, for example, polycaprylactones and polylactic-co-glycolic acids (PLGA's)).

In some embodiments, the TWEAK-R agonist is administered via an implantable system. Implantable systems can include rods and discs, and can contain excipients such as PLGA and polycaprylactone.

Oral delivery systems for the TWEAK-R agonist include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems for the TWEAK-R agonist include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

The TWEAK-R agonist may, for example, be placed into sterile, isotonic formulations with or without cofactors which stimulate uptake or stability. The formulation is preferably liquid, or may be lyophilized powder. For example, the TWEAK agonist may be diluted with a formulation comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (U.S.P).

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeably polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers, 22: 547-56 (1985)); poly(2-hydroxyethyl-methacrylate) or ethylene vinyl acetate (Langer et al., J. Biomed. Mater. Res., 15: 167-277 (1981); Langer, Chem. Tech, 12: 98-105 (1982)).

Methods of the invention also include delivery of an effective amount of a TWEAK-R agonist to isolated, cultured pancreatic cells to induce the expansion of said cells in vitro.

The methods, formulations, and dosages of the invention may be evaluated in a known model of diabetes. These models include animal models such as the mouse model described in the following examples. When testing the compositions of the invention in animal models, the biological therapeutic agent should have activity in the animal. For example, a mouse agonist anti-TWEAK-R antibody may be used in lieu of a human antibody if the human antibody does not cross-react with mouse TWEAK-R.

The following examples provide illustrative embodiments of the invention. The skilled artisan will recognize the numerous modifications and variations that may be performed without altering the spirit or scope of the present invention. Such modifications and variations are encompassed within the scope of the invention. The examples do not in any way limit the invention.

### EXAMPLES

### Example 1

### TWEAK Overexpression Induces Pancreatic Ductal Cell Hyperplasia

Novel strategies to expand precursor cells in the pancreas would constitute a significant advance towards treatment of diabetes, which result from an inadequate amount of insulin-producing 13-cells. TWEAK is a member of the TNF superfamily of cytokines that mediates pleiotropic effects, including proinflammatory activities, angiogenenesis, and the regulation of cell survival, proliferation and death, through its receptor TWEAK-R (FGF-inducible molecule 14; Fn14). TWEAK-R is expressed by epithelial and mesenchymal cells and signals via the NE-κB, MAPK and AKT pathways. Interestingly, TWEAK-R expression is normally expressed at relatively low levels and is highly upregulated in contexts of tissue injury and regeneration, and chronic inflammatory disease, supporting a physiological role for this pathway in coordinating acute inflammation and tissue repair and pathological role in chronic inflammatory disease. TWEAK-R is expressed by a variety of progenitor cell types, including biliary duct-associated liver progenitor cells, mesenchymal stem cells, as well as skeletal muscle, cartilage, bone, adipocyte and neuronal progenitors. In the liver, TWEAK induced expansion of duct-associated progenitors. Although the identity of pancreatic progenitors is still unknown, some previous studies have suggested that pancreatic progenitors derive from the duct epithelium. We are currently investigating whether the TWEAK/TWEAK-R pathway plays an important role in regulating pancreatic progenitors and regeneration.

To determine whether increased levels of TWEAK have an effect on regeneration of pancreatic cells, TWEAK was overexpressed in mice using an adenoviral expression vector, and pancreas samples from the mice were then observed histologically. An adenovirus containing the coding sequence for murine TWEAK was constructed by inserting the soluble TWEAK-encoding sequence into a replication defective adenoviral vector under the control of the constitutive CMV promoter. Recombinant adenoviral particles containing the TWEAK ORF under the control of the constitutive CMV promoter, referred to as “adeno-TWEAK”, were created by standard methods (Ng, P., et al., Hum. Gene Ther., 11: 693-699 (2000); and Ng, P., et al., Hum. Gene Ther., 10: 2667-267 (1999)). Briefly, the first generation adenoviral vector (E1, E3 deleted, serotype 5) expressing soluble murine TWEAK was created via standard methods in 293 cells (ibid). The vector was purified using double-cesium chloride density equilibrium gradient centrifugation and was then stored at −80° C. (in 10 mM TrisHCl, 1 mM MgCl2, 10% glycerol vol/vol, pH 8.0). Expression of murine TWEAK from this vector was verified using an enzyme-linked immunosorbent assay (ELISA) both in vitro with A549 cells (ATCC) and in vivo with mice given the vector intravenously. A control adenovirus containing the coding sequence of GFP, referred to as “adeno-GFP,” was constructed in the same manner. The TWEAK-encoding adenovirus was delivered to wild-type C57BI/6 female mice of 6-8 weeks of age purchased from Taconic Farms or Jackson Laboratories. A total adenoviral vector dose of 10¹¹ viral particles was used in both control and experimental mice in order to ensure a good transduction efficiency. Serum TWEAK levels were typically about 300 ng/ml 8 days after injection, 50 ng/ml 14 days after injection, and 27 ng/ml 27 days after injection. Three weeks after injection, pancreases from euthanized mice are isolated, fixed, sectioned, and stained for the Ki-67 protein, a cellular marker for proliferation.

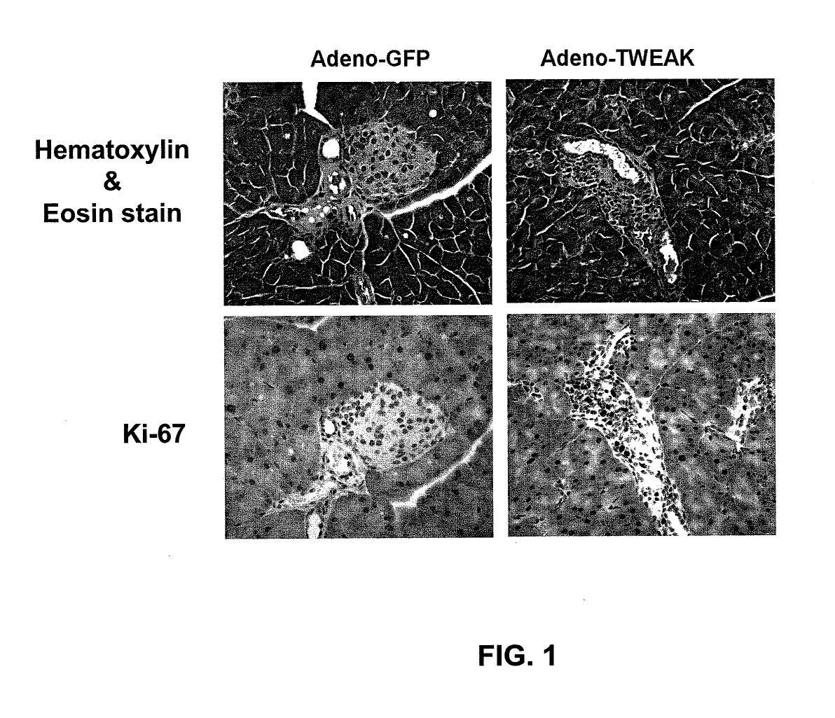

As shown in FIG. 1, the number of proliferating cells in periductal areas of the pancreas is far greater in mice infected with the adeno-TWEAK than in control mice infected with adeno-GFP.

### Example 2

### TWEAK-R is Expressed on Pancreatic Duct-Derived Cells

The expression levels of TWEAK-R were measured by mRNA microarray analysis in pancreatic duct cells isolated from normal rat pancreas or 2¾ days after partial pancreatectomy. Microarray analysis was performed using standard procedures (see Flamez et al., Diabetes, 51: 2018-2024 (2002); and Webb, et al., Proc Natl Acad Sci USA, 97: 5773-5778 (2000)). The normal pancreatic and post-pancreatectomy duct cells produced detectable TWEAK-R mRNA, while isolated pancreatic islets did not.

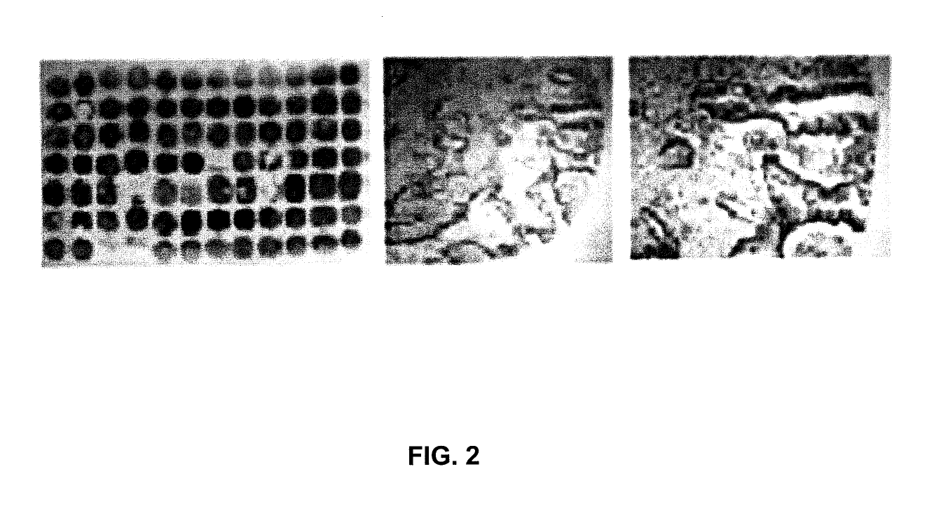

TWEAK-R is expressed on a high frequency of pancreatic adenocarcinomas (Han et al., Cancer Res., 62(15): 4532 (2002)), which are believed to originate from ductal cells. FIG. 2 shows the expression of TWEAK-R in human pancreatic tumors, detected by immunohistochemical staining of a human pancreatic tumor tissue microarray. Sixteen of 42 tissue samples (42%) were positive for TWEAK-R.

### Example 3

### Fc-TWEAK Induces Proliferation of Cells in Pancreatic Duct Epithelium and Ductal Adjacent Regions

Eight- to ten-week old adult C57BI/6 female mice were injected with 200 μg of Fc-TWEAK or control protein P1.17 in either a single injection (acute treatment), or twice per week following an initial injection (chronic treatment; injections were performed at day 0, 3, 7, 10, and 14). Pancreatic tissue was surgically obtained from Ketamine/Xylazine-anesthetized mice at various time points and mice were sacrificed immediately after pancreatic tissue was removed. Mice did not receive an injection of Fc-TWEAK or P1.17 on the day of sacrifice. Tissue samples were fixed, sectioned, and immunostained for the proliferation marker Ki-67.

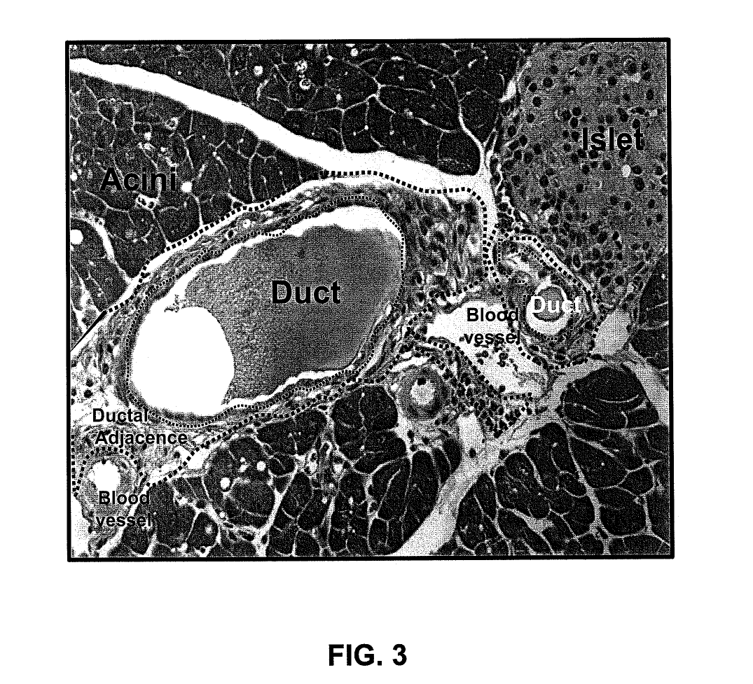

FIG. 3 shows a representative hematoxylin and eosin (H & E) stained mouse pancreas cross section. The pancreas consists of acini, ducts, and islets. Cells around the pancreatic duct which do not show the typical structure of acinar, islet, or blood vessel cells are referred to as ductal adjacent cells.

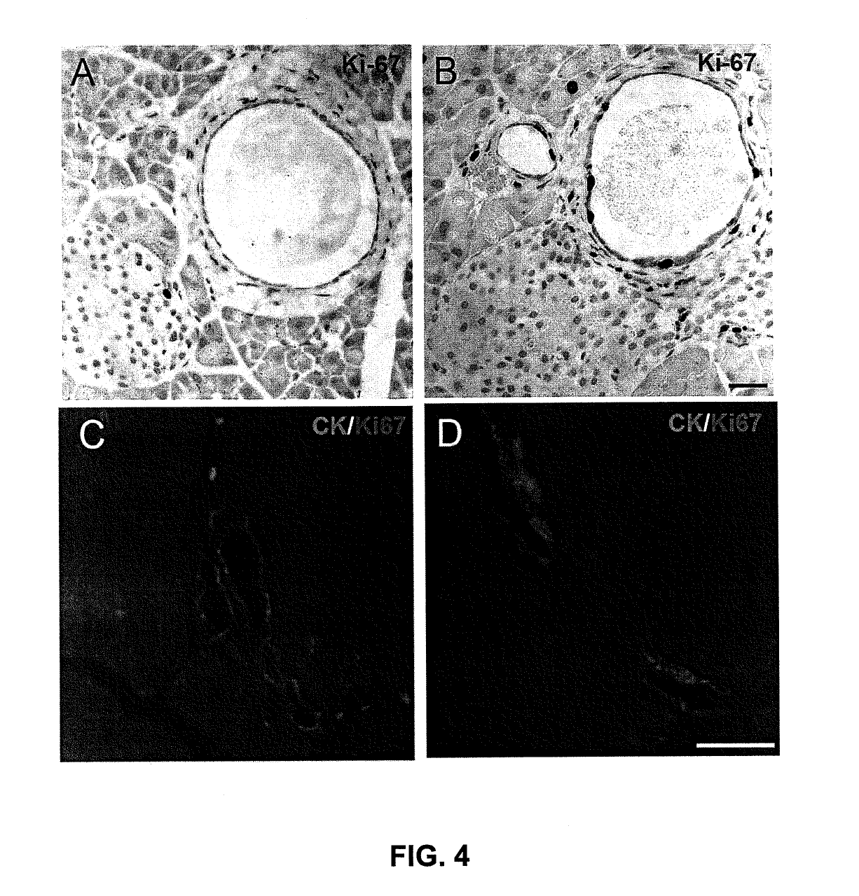

FIG. 4 contains representative images showing the increased proliferation of cells in the pancreatic ductal region three days after Fc-TWEAK treatment, as evidenced by Ki-67 staining (scale bars=200 μm). FIG. 4A shows the proliferation present in the ductal region following treatment with the control protein P1.17, while FIG. 4B shows that an increased number of proliferating cells are present in the same region in mice receiving Fc-TWEAK for the same length of time. FIGS. 4C and D show pancreatic tissue constained for ductal epithelial marker CK and Ki-67 on day four after control (FIG. 4C) or Fc-TWEAK (FIG. 4D) treatment. Overlap of CK and Ki-67 staining in the TWEAK-treated cells demonstrates proliferation of ductal epithelial cells.

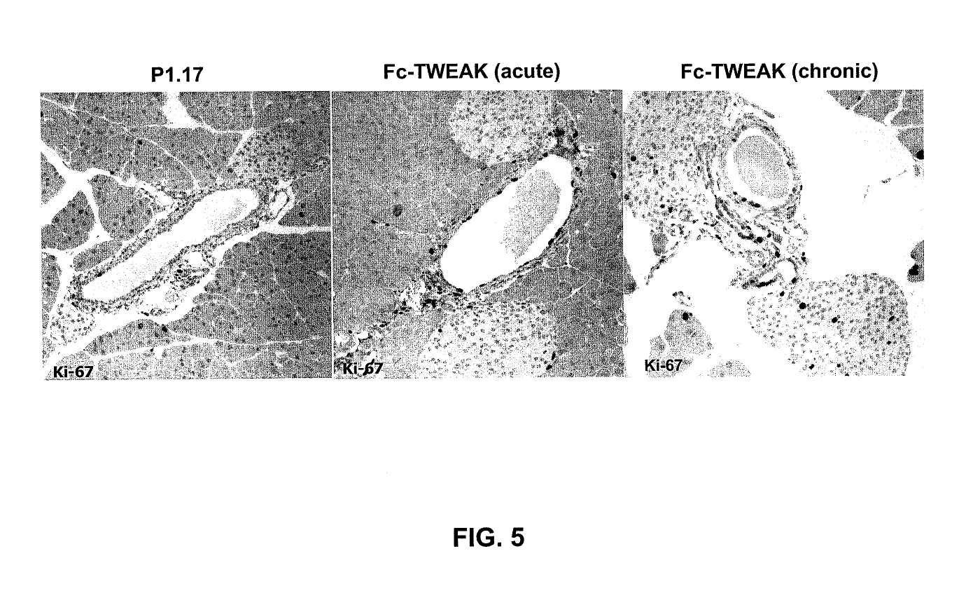

The increase in proliferating cells in the ductal region was similar on day 4 after either acute Fc-TWEAK treatment or chronic Fc-TWEAK treatment (FIG. 5). These results are representative of four mice per treatment group.

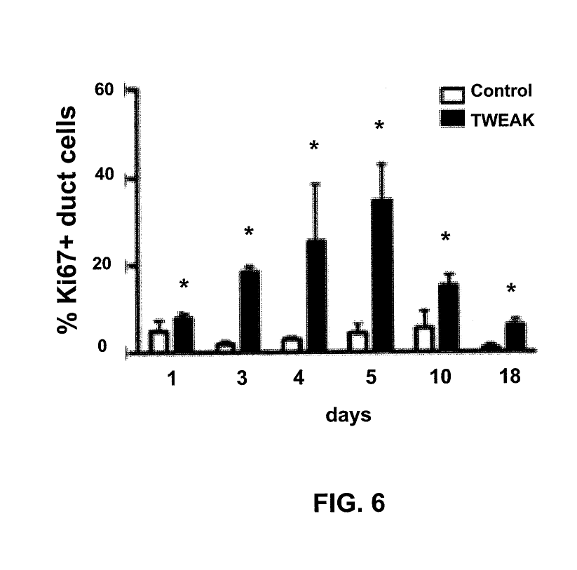

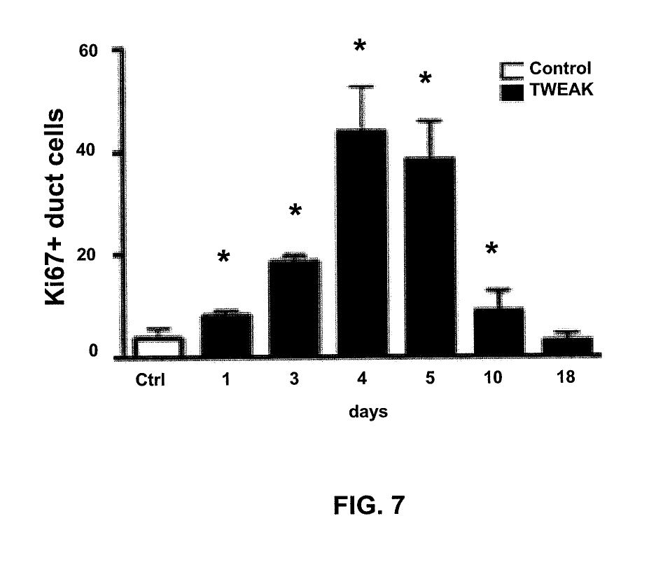

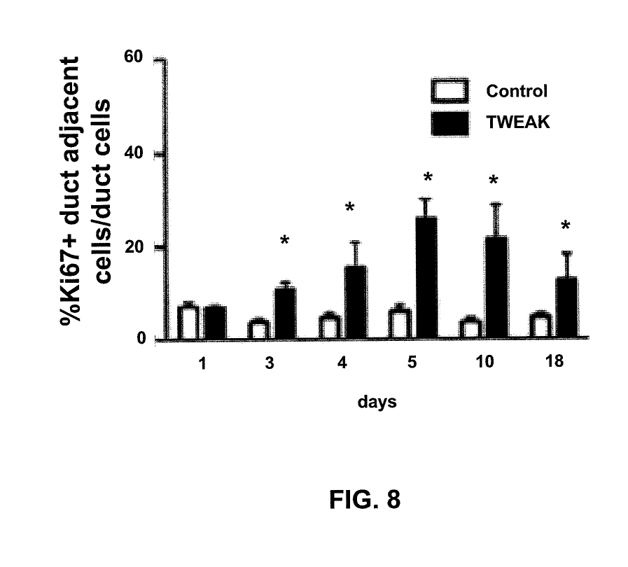

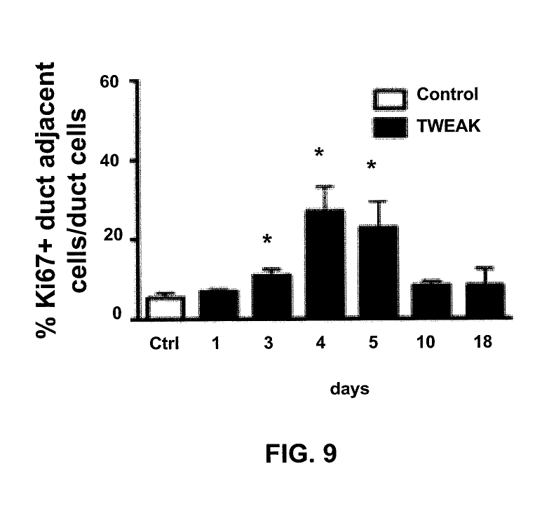

The percentage of ductal cells that were positive for Ki-67 after Fc-TWEAK or P1.17 treatment for 1, 3, 4, 5, 10, and 18 days was calculated by counting the number of Ki-67 positive ductal cells and the total number of ductal cells in multiple fields of a given section for each individual animal. These percentages are shown in FIG. 6 for mice given chronic Fc-TWEAK treatment and in FIG. 7 for mice given acute Fc-TWEAK treatment as compared to treatment with control Ig protein P1.17. (Data are shown as mean±Standard error of the mean [SEM; n=4].) With both treatment regimens, the number of Ki-67-positive duct epithelial cells was increased in Fc-TWEAK-treated versus control Ig-treated mice. The percentage of cells in ductal adjacent regions that stained positively for Ki-67 was also quantified by counting the number of Ki-67 positive ductal adjacent cells and the total number of ductal cells per duct in multiple fields of a given section for each individual animal, and is shown in FIG. 8 for mice given chronic Fc-TWEAK treatment and FIG. 9 for mice given acute treatment. (As in FIGS. 6 and 7, data are shown as mean±SEM [n=4].) As was observed for duct epithelial cells, Fc-TWEAK treatment increased the number of proliferating cells in ductal adjacent regions relative to treatment with control Ig. Chronic Fc-TWEAK treatment was required for sustained proliferation of cells in duct-associated regions at longer time points (day 10 and day 18). Measurements showing significant induction of proliferation relative to their respective controls (p<0.05) in FIGS. 6-9 are indicated by an asterisk.

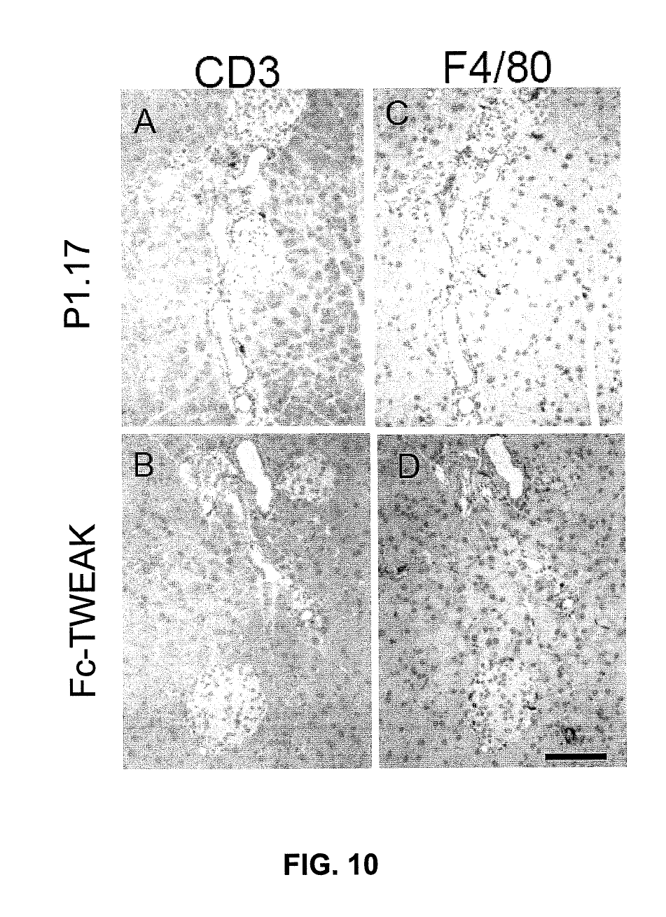

TWEAK treatment does not induce generalized pancreatic inflammation. FIG. 10 contains representative images showing that immunostaining of CD3 (T-cell marker) and F4/80 (macrophage marker) was not elevated on pancreas sections from mice on day four after chronic TWEAK treatment relative to control (scale bar=200 μm). Similar results were obtained in all the time points performed (day 1, 3, 4, 5, 10 and 18) under either the acute or chronic treatment regimen.

### Example 4

### The Mitogenic Effect of TWEAK on Pancreatic Cell Proliferation in the Ductal Regions is Mediated Through TWEAK-R

Generation of TWEAK-R KO mice was described (Jakubowski et al., J. Clin. Invest. 115:2330-40 (2005)). In brief, a 10-kb Kpn1 genomic DNA fragment containing the full murine TWEAK-R gene was isolated, and a targeting vector was designed to delete the first 2 exons, which contained the entire extracellular ligand-binding domain of TWEAK-R. The target vector was transfected into the J1 129 ES cell line and selected with G418. ES cell clones were screened for homologous recombination using Southern blot, and the correct clones were injected into C57BU6 blastocysts to generate chimeras. Mice heterozygous for targeted TWEAK-R alleles were obtained through further breeding and identified using Southern blot or PCR. The null mutation was confirmed by both Northern blot and RT-PCR. Mice were bred to homozygosity on the 129 background. The TWEAK-R mutation was backcrossed 5 times onto the C57BU6 background under SPF conditions.

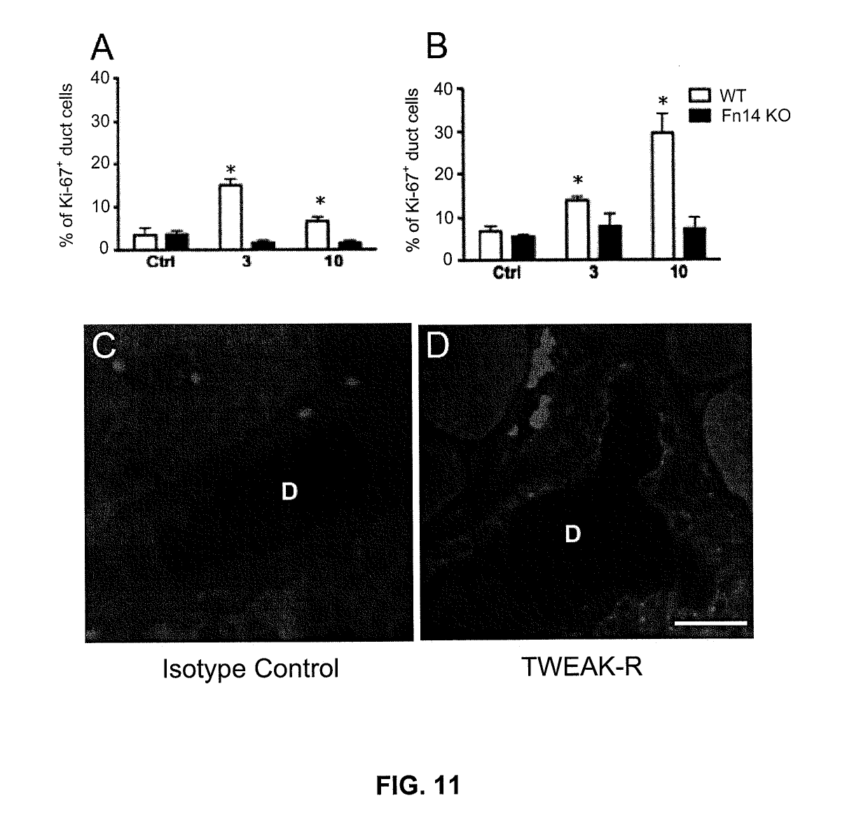

TWEAK-R KO and wild type mice were treated with control protein P1.17 or TWEAK twice per week for a duration of three or ten days. Pancreatic sections from the mice were immunostained for Ki-67. FIG. 11 shows the number of Ki-67-positive ductal cells (FIG. 11A) or ductal adjacent cells (FIG. 11B) counted in the sections. Data are shown as the mean±SEM (n=4). An asterisk indicates a p value of less than 0.05 between TWEAK-treated and control samples. While the percentage of Ki-67-positive ductal and ductal adjacent cells was greater in pancreas sections from WT mice treated with TWEAK than in mice treated with control protein, there was no increase in Ki-67 positive cells in TWEAK-R KO mice treated with TWEAK relative to mice treated with control protein. Thus, TWEAK-R is required for TWEAK's proliferative effect on pancreatic ductal epithelial cells.

Moreover, TWEAK-R is specifically expressed in pancreas ductal epithelium in TWEAK-treated mice, as evident on TWEAK-R-immunostained (FIG. 11D) or isotype control-immunostained (FIG. 11C) pancreas sections from mice on day three after TWEAK treatment (Scale bar=100 μm).

### Example 5

### TWEAK Induces Expansion of Cultured Human Pancreatic Ductal Cells

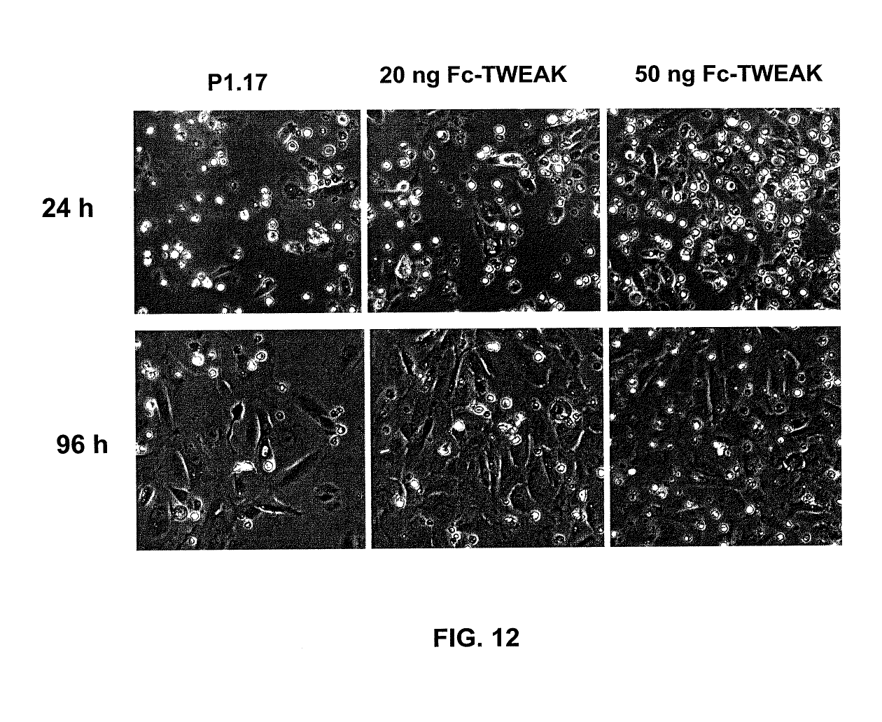

Recombinant TWEAK was added to cultures of purified human ductal cells (positive for CA19-9, a serologic marker of pancreatic cancer) during the expansion phase of growth following isolation. Human ductal cells were isolated as described by Yatoh et al (Diabetes, 56: 1802-9 (2007)). In brief, human pancreatic tissue was digested and islets isolated by standard methodology of Ricordi (E. Linetsky et al., Diabetes, 46: 1120-23 (1997)), after which the remaining tissue was allowed to settle in conical tubes. The supernatant was then aspirated to remove low-density components including dead cells, and the remaining cells were dispersed with a trypsin/EDTA solution to obtain single cells which were designated “crude” or unpurified duct cells. Purification of duct cells from this crude cell preparation was then achieved by immunomagnetic sorting with an antibody specific for CA19-9, a carbohydrate marker present on cells throughout the human ductal tree. Purified duct cells were plated in non-tissue culture T flasks where they were expanded in media without or with recombinant soluble TWEAK added at the initiation of the culture. The TWEAK was prepared as previously described (Jakubowski et al., J. Cell Science, 115: 267-74 (2002)). The cell media was changed to new media with or without TWEAK on day three, and on day one and four, the cells were photographed, counted by hemocytometry, and mRNA was extracted for further study. In tissue from four pancreases, samples exposed to TWEAK (20 or 50 ng/ml) had more robust cell numbers than those of the same pancreas not exposed to TWEAK, as evident in the representative photos in FIG. 12.

This finding indicates that at least some of the population of purified human ducts is TWEAK-responsive, possibly mediated through TWEAK-R after immunomagnetic sorting, which is up to 36 hours after the pancreas was removed from the organ donor. Purified CA19-9-positive cell populations contain no insulin-positive cells after sorting, as tested by immunostaining or by RT-PCR. However, these cells can become ductal tubules with 1% insulin-positive cells 4 weeks after engraftment under the kidney capsule of NODscid mice (Yatoh et al. Diabetes, 56:1802-1809 (2007)), indicating that pancreatic progenitor cells are present in these cell populations.

### Example 6

### Fc-TWEAK Induces the Expression of Pancreatic Progenitor Markers

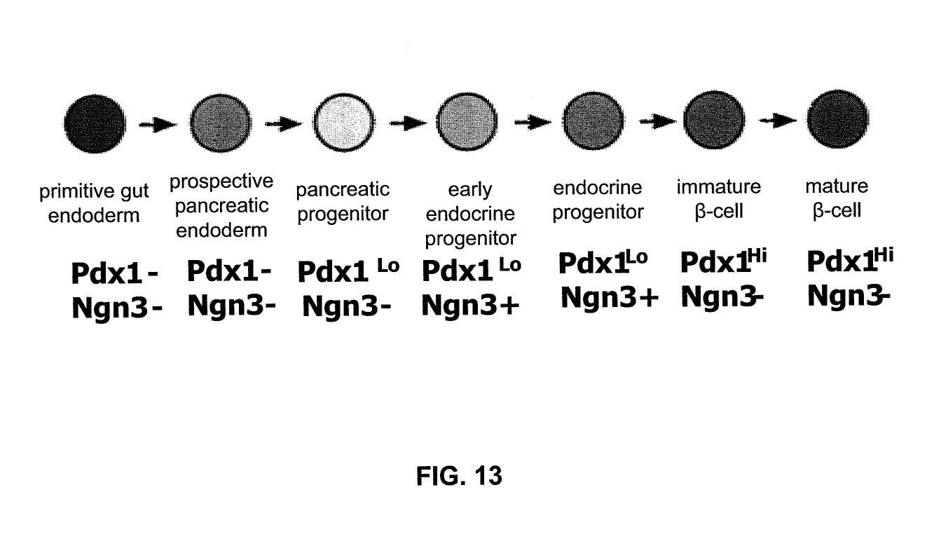

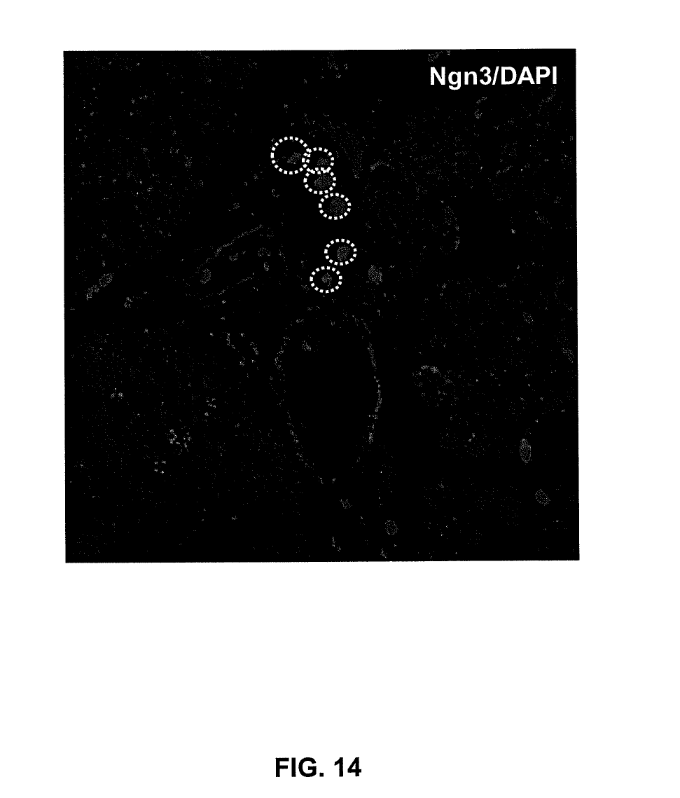

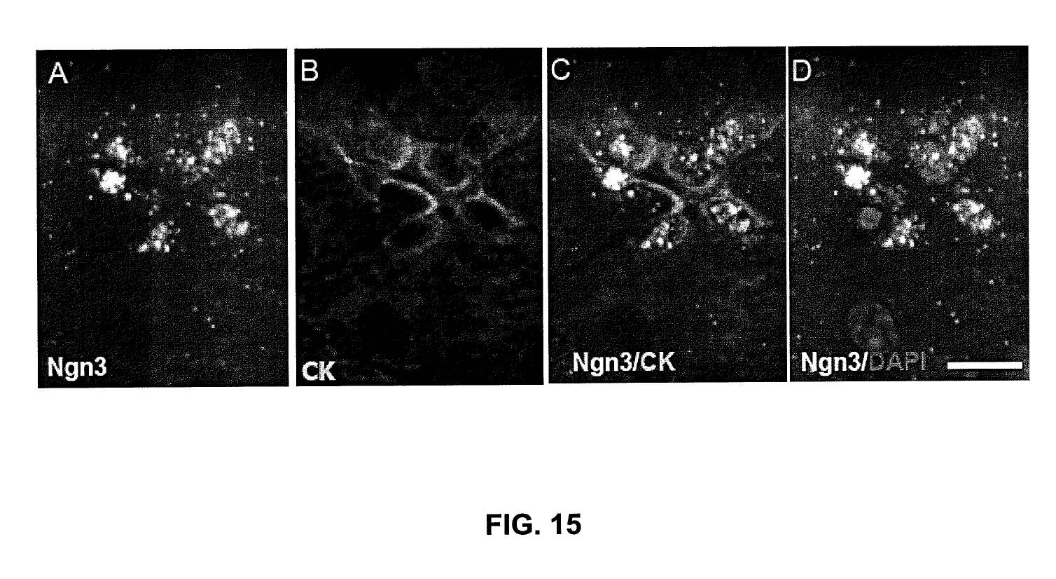

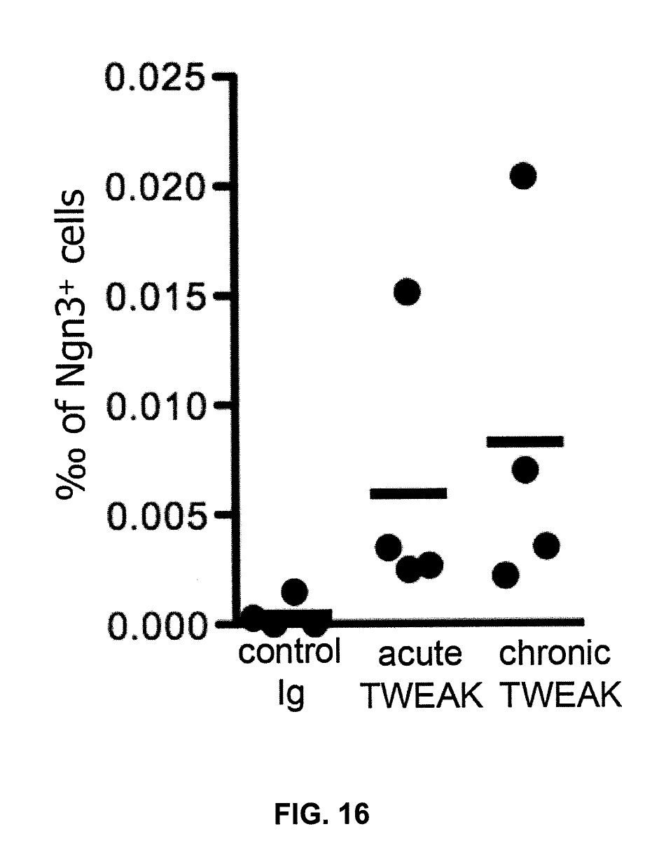

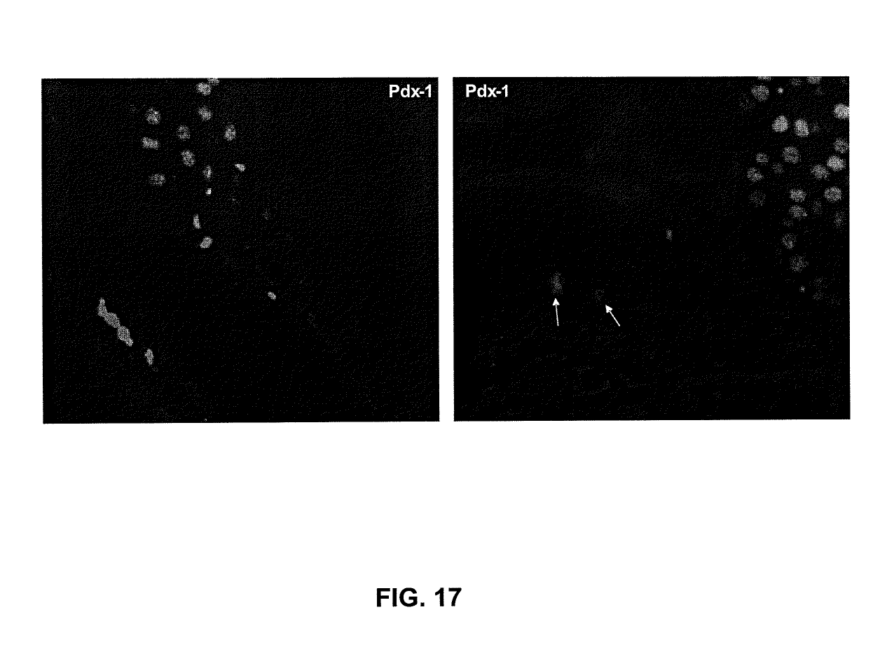

FIG. 13 shows the dynamic expression of the transcription factors Ngn3 and Pdx1, which regulate the lineage specification of pancreatic progenitors during mouse embryonic development. Pdx1 is expressed at low levels in pancreatic progenitor cells and at high levels in islet β cells, while Ngn3 is expressed transiently in endocrine progenitor cells but didappear postnatally. Pancreatic tissue samples from adult mice treated with Fc-TWEAK were obtained as described in Example 3 and immunohistochemically stained for Ngn3, ductal epithelial marker CK, and DNA (DAPI). Rare Ngn3-positive cell clusters were evident on day four after either chronic (FIG. 14) or acute (FIG. 15) Fc-TWEAK treatment in the ductal regions. (FIG. 15A shows Ngn3 staining, FIG. 15B shows CK staining, FIG. 15C shows Ngn3 and CK costaining, and FIG. 15D shows Ngn3 and DAPI costaining.) FIG. 16 shows the frequency of Ngn3-positive cells in these samples, determined by dividing the number of Ngn3-positive cells observed in a single section by the total number of cells counted in that section. Each point in FIG. 16 is the average of two random full footprint pancreatic sections from an individual animal, with the horizontal bar indicating the mean of four mice/group. Fc-TWEAK also induced a low expression level of Pdx1 in ductal epithelial cells, as shown in FIG. 17. The induction of cells expressing endocrine lineage specification factors by Fc-TWEAK treatment indicates that TWEAK increases the frequency of cells with pancreatic progenitor potential.

### Example 7

### Chronic Fc-TWEAK Treatment for 18 Days Mimics the Response to Partial Pancreatectomy

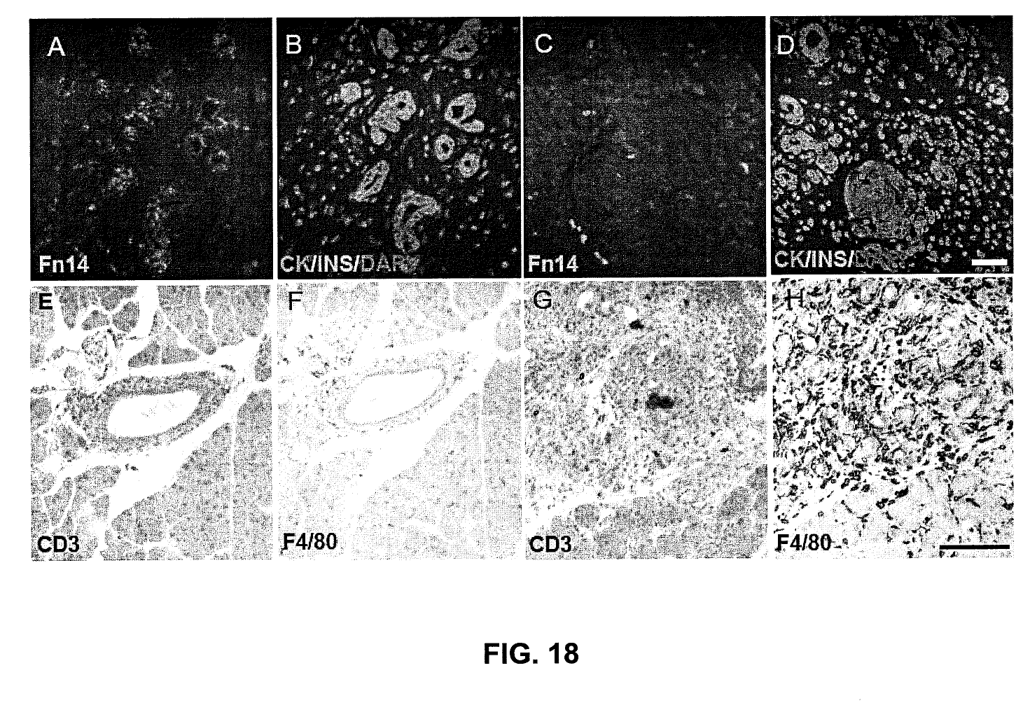

Eight week-old mice were subjected to partial pancreatectomy, and the pancreas was excised four days after surgery. FIG. 18 shows representative images of pancreatic serial sections from eight mice on day four after partial pancreatectomy stained with TWEAK-R (FIGS. 18A and C) or CK, insulin, and DNA (DAPI) (FIGS. 18B and D). FIGS. 18A and B show representative images of immature regenerating foci, and FIGS. 18C and D show representative images of mature foci. Serial sections of pancreas from sham-operated mice (FIGS. 18E and F) and from mice four days after Px (FIGS. 18G and H) were stained with T-cell marker CD3 (FIGS. 18E and G) and macrophage marker F4/80 (FIGS. 18F and H). CD3-positive T cells and F4/80-positive macrophages were induced specifically in the regenerating foci after partial pancreatectomy compared with the low expression of CD3 and F4/80 in the sham-operated pancreas.

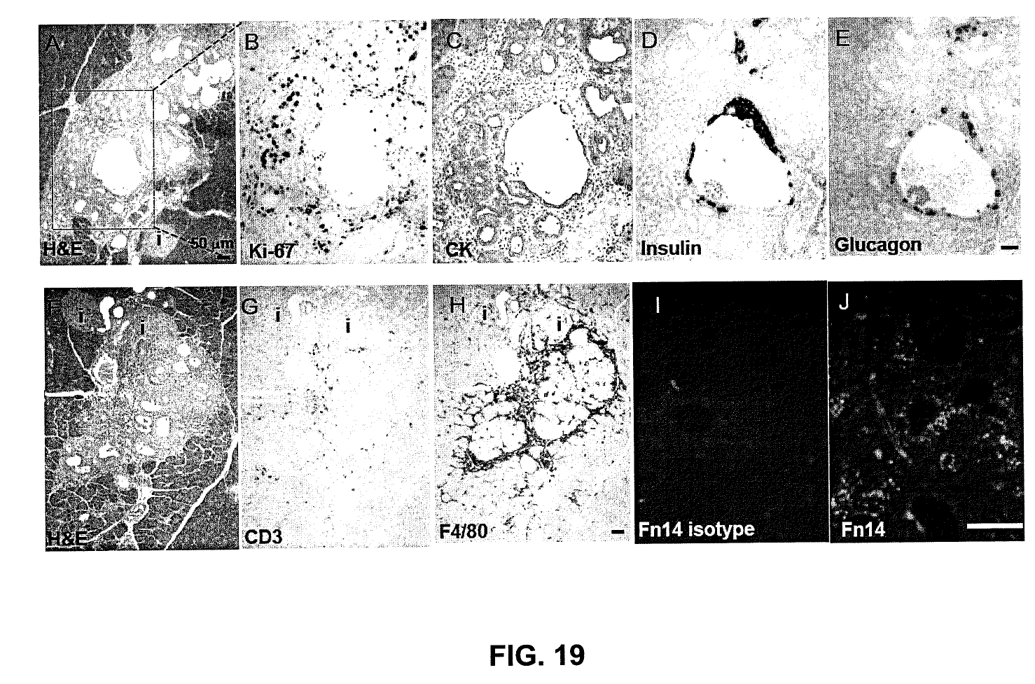

Regeneration foci were also identified on day 18 after TWEAK treatment twice weekly in 4/4 animals (FIG. 19A, representative H&E stained pancreas section at low magnification showing the overall regenerating area). Regeneration foci were not observed in control mice. Serial sections of the TWEAK-treated mice were stained for Ki-67 (FIG. 19B), CK (FIG. 19C), insulin (FIG. 19D), glucagon (FIG. 19E). Serial sections from another Fc-TWEAK treated pancreas were stained with H&E (FIG. 19F), CD3 (FIG. 19G), F4/80 (FIG. 19H), and other serial sections were stained with TWEAK-R (FIG. 19J) and isotype control (FIG. 19I). The islets were labeled as “i”. Scale bars=50 μm. Therefore, the focal areas of regeneration induced by 18 days chronic TWEAK treatment mimic those observed after partial pancreatectomy, including highly proliferating ductules surrounded by mesenchymal cells, some hormone-expressing duct cells as well as scattered hormone-expressing cells (insulin or glucagon) and increased CD3 and F4/80 expression.

### Example 8

### The TWEAK-R/TWEAK Pathway is Important for Pancreatic Regeneration after Partial Pancreatectomy

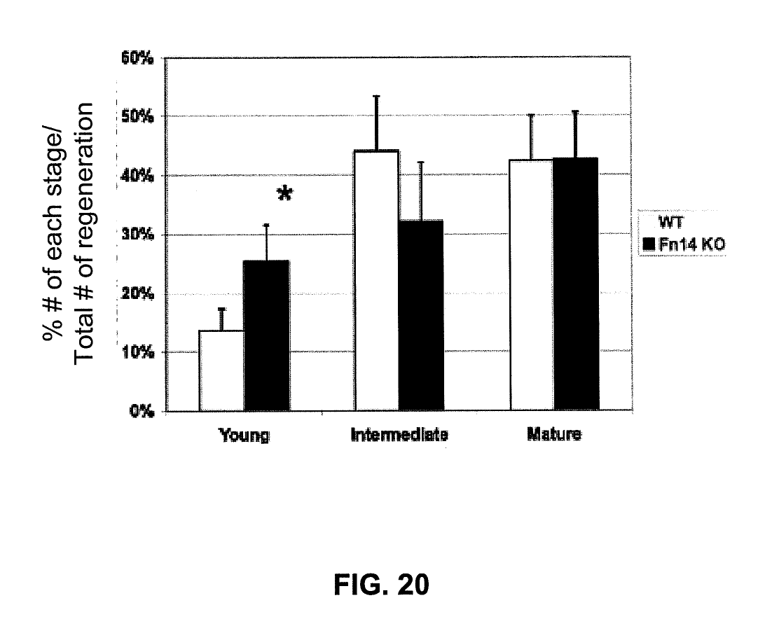

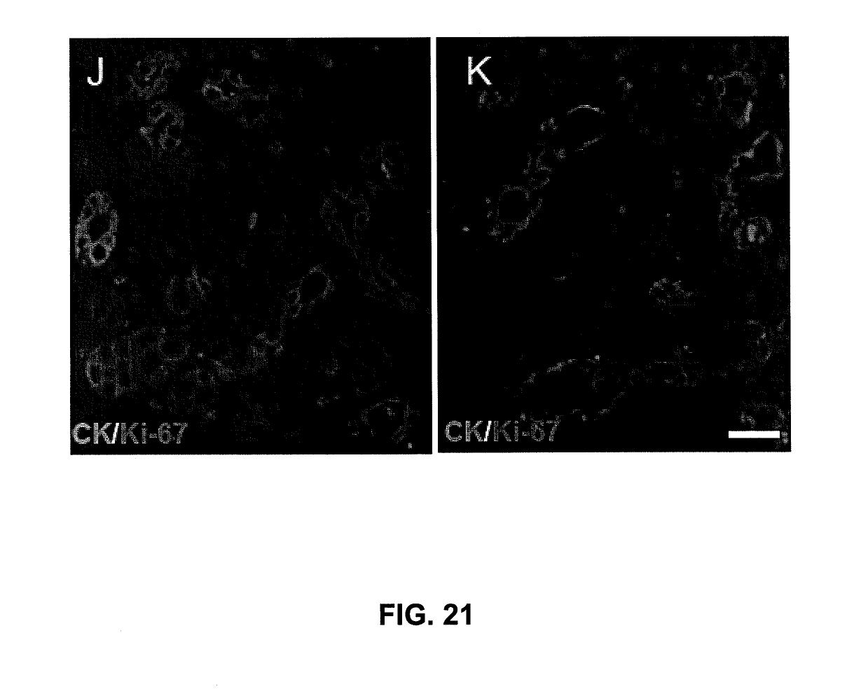

To examine the role of the TWEAK-R/TWEAK pathway in pancreatic regeneration after pancreatectomy, regeneration was examined in wild type and TWEAK-R KO mice on day four following pancreatectomy. FIG. 20 shows the percentage of regenerating foci at early (“young”), intermediate, and mature stages in wild type mice (white bars) and TWEAK-R KO mice (black bars) four days after partial pancreatectomy. The number and stage of regenerating foci was assessed on at least two sections per animal. Sections were at least 200 μm apart, and six animals were in each group. No regenerating foci were identified in sham-operated mice. (Data in FIG. 20 are shown as the mean±SEM, and asterisk indicates a p value of less than 0.05 comparing TWEAK-R KO to wild type mice.) The number of regenerating foci at the young stage was increased in TWEAK-R KO mice relative to wild type mice. Pancreatic sections costained for CK and Ki-67 from TWEAK-R KO and wild type mice four days after partial pancreatectomy showed that the appearance of Ki-67-positive ductal epithelial cells was diminished in TWEAK-R KO mice (FIG. 21B) compared to wild type mice (FIG. 21A; scale bar=200 μm).

### Example 9

### Determination that TWEAK Treatment can Give Rise to Differentiating and/or Fully Differentiated Pancreatic Cell Types That are Derived from Cells with Progenitor Potential

Analysis of the proliferation of specific cell types within the pancreatic tissue of mice treated with Fc-TWEAK using sections double stained for Ki-67 and various cell lineage markers determines the identity of proliferating cell types. Coexpression of Ki-67 and pancreatic progenitor markers indicates that TWEAK induces the expansion of progenitor cells. However, since Ki-67 is only expressed during the cell cycle, progenitor cells that differentiate from Ki-67 positive cells may express progenitor markers and no longer express Ki-67. Therefore, an increase in the number of cells expressing pancreatic progenitor markers in the pancreatic tissue of Fe-TWEAK treated mice is indicative that TWEAK treatment increases the frequency of cells with pancreatic progenitor potential, as shown in Example 6.

Furthermore, the ability of TWEAK-induced cells to differentiate is determined by the identification of “transitional cells” coexpressing markers that are normally characteristic of different cell types, indicating transition from one cell type into another cell type. Examples of such markers include but are not limited to coexpressed ductal and progenitor markers, ductal and hormonal markers such as insulin, and progenitor and hormonal markers. The ability of TWEAK-induced cells to differentiate can also be determined by the appearance of newly formed focal areas of proliferating ductules, within which regions scattered hormone-positive cells or clusters of hormone-positive cells, such as insulin-positive cells, are found. The ability of TWEAK-induced cells to differentiate into mature pancreatic cells types, such as islet β cells, can also be determined by lineage-tracing studies.

Exemplary markers for use in this analysis include progenitor markers, such as, e.g., Pdx-1—a marker of early foregut and pancreatic progenitors that is required for development of all pancreatic cell types (Jonsson et al., Nature, 371: 606-609 (1994); Offield et al., Development, 122: 983-985 (1996)), and is expressed in normal adult animals in islets and in replicating ducts (Sharma et al., Diabetes, 48: 507-513 (1999)); nestin (Seaberg et al., Nat Biotechnol, 22: 1115-1124 (2004)); c-met (Suzuki et al., Diabetes, 53: 2143-2152 (2004)); E-cadherin, β-catenin, and notch components (Jensen et al., Gastroenterology, 128: 728-741 (2005)); Hes-1, a marker of early pancreatic progenitor cells that in normal adult pancreas is expressed by centroacinar cells and some duct cells (Stanger et al., Cancer Cell, 8: 185-195 (2005)); Ngn3, a transiently expressed transcription factor only detected in pancreatic progenitor cells that do not express endocrine hormones (Gradwohl et al., Proc. Natl. Acad. Sci. U.S.A, 97: 1607-1611 (2000)).

Other suitable markers include (1) ductal markers, such as, e.g., Dolichos biflorus agglutinin (DBA) lectin, which marks the whole ductal tree from the common bile/pancreatic duct through to the centroacinar cells and some embryonic pancreatic progenitor cells; cytokeratins CK-19 and CK-20; carbonic anhydrase II; and mucin 1; (2) acinar markers, such as, e.g., amylase; (3) endocrine cell markers, such as, e.g., insulin, glucagon, somatostatin, and pancreatic polypeptide; (4) oval cell markers such as, e.g., alpha-fetoprotein, cytokeratin 19, albumin, and c-kit; and (5) non-pancreatic cell markers, such as, e.g., CD45 (hematopoietic cells) and alpha-SMA (stromal cells).

The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1-141. (canceled)

142. A method for inducing regeneration of pancreatic tissue in a subject, comprising administering to a subject in need thereof a therapeutically effective amount of a TWEAK receptor (TWEAK-R) agonist sufficient to induce regeneration of pancreatic tissue.

143. The method of claim 142, wherein pancreatic tissue is selected from the group consisting of pancreatic islet tissue and beta islet cells.

144. The method of claim 142, wherein the subject has type 1 or type 2 diabetes, and the method comprises administering to the subject an amount of a TWEAK-R agonist effective for inducing the regeneration of insulin-secreting pancreas tissue.

145. The method of claim 142, wherein the subject has a diabetes related condition selected from the group consisting of syndrome x (metabolic syndrome), insulin resistance, nephropathy, ketoacidosis, hyperosmolar hyperglycemic nonketoic syndrome, gastropareseis, and diabetic kidney disease.

146. The method of claim 142, wherein the subject has undergone removal of pancreatic tissue.

147. The method of claim 142, wherein the subject has pancreatic cancer.

148. The method of claim 142, wherein the subject has received a cell or tissue transplant in the pancreas, wherein the transplant comprises progenitor cells capable of differentiating into pancreatic cells, pancreatic progenitor cells, or cells capable of dedifferentiating into pancreatic progenitor cells.

149. The method of claim 142, wherein the subject has undergone a partial pancreatectomy.

150. A method of treating diabetes in a subject, comprising administering to a subject with diabetes i) a cell or tissue transplant comprising progenitor cells capable of differentiating into pancreatic cells, and ii) a therapeutically effective amount of a TWEAK-R agonist sufficient to induce regeneration of pancreatic tissue.

151. The method of any one of claims 142-150, further comprising co-administering to the subject an anti-inflammatory or immunomodulatory agent.

152. A method of treating diabetes comprising the steps of:
a) culturing pancreatic progenitor cells in vitro, wherein said progenitor cells are isolated from a subject with diabetes, totipotent stem cells, pluripotent stem cells, adult stem cells, or embryonic stem cells;
b) inducing the proliferation of said cultured progenitor cells with an effective amount of a TWEAK-R agonist to generate an expanded population of said progenitor cells; and
c) transplanting said expanded population of progenitor cells into the pancreas of the subject with diabetes;
wherein insulin-producing pancreatic cells are regenerated in the subject from said transplanted progenitor cells.

153. The method of any of claims 142-150 and 152, wherein the TWEAK-R agonist is selected from the group consisting of: TWEAK, a TWEAK fusion protein, a TWEAK analog, a TWEAK mimetic, and an agonistic TWEAK-R antibody.

154. The method of claim 153, wherein TWEAK is a polypeptide with an amino terminus at any position between amino acids 46 and 104 of SEQ ID NO: 1 or SEQ ID NO: 2 and a carboxy terminus at amino acid 249 of SEQ ID NO: 1 or SEQ ID NO: 2.

155. The method of claim 153, wherein the TWEAK analog is a polypeptide that is at least 80% identical to SEQ ID NO: 1 or SEQ ID NO: 2.

156. The method of claim 153, wherein the TWEAK fusion protein comprises a polypeptide that is at least 80% identical to of SEQ ID NO: 1 or SEQ ID NO: 2 and an Fc portion of an immunoglobulin.
